(19) United States
(12) Patent Application Publication
(10) Pub. No.: US 2006/0166195 A1
(43) Pub. Date: Jul. 27, 2006

(54) Method for detecting and identifying the presence of biological materials derived from fish and oligonucleotides therefor

(76) Inventors: Carole Donne-Gousse, Guillaume (FR); Vincent Laudet, Lyon (FR); Catherine Hanni, Lyon (FR)

Correspondence Address: YOUNG & THOMPSON, 745 SOUTH 23RD STREET, 2ND FLOOR, ARLINGTON, VA, 22202, US

(21) Appl. No.: 10/480,584

(22) PCT Filed: Jun. 13, 2002
(86) PCT No.: PCT/FR02/02031

§ 371 Date: Aug. 4, 2004

(30) Foreign Application Priority Data
Jun. 13, 2001 (FR) 01/07735

(51) Int. Cl. C12Q 1/68

(52) U.S. Cl. 435/6

(57) ABSTRACT

A subject of the present invention is a method for detecting the presence of biological materials originating from gadiformes chosen from the group constituted by the gadidae, the merluccidae, the macrouridae and/or the moridae, in a sample of organic material, characterized in that the presence of mitochondrial DNA originating from gadiformes chosen from the group constituted by the gadidae, the merluccidae, the macrouridae and/or the moridae in said organic material is determined by amplification of at least one sequence or fragment of mitochondrial DNA specific to the genome of the gadiformes chosen from the group constituted by the gadidae, the merluccidae, the macrouridae and/or the moridae, and contained in the mitochondrial DNA extracted from said sample, namely at least one sequence or fragment present in the genomes of the gadiformes chosen from the group constituted by the gadidae, the merluccidae, the macrouridae and/or the moridae, but absent from the genomes of the other animal genera, and in particular of the other animal species.

A subject of the present invention is a method for detecting and identifying the presence of biological materials originating from gadiformes, in a sample of organic material.

It also has as a subject the oligonucleotides for implementing this method, and the DNA fragments obtained using said oligonucleotides.

The increasing economic value of certain animal species used for human food is linked to a major imbalance between supply and demand in the market. A direct consequence of this is the introduction of an increase in the practice of food adulteration. One of the most common ways of adulterating food comprises the substitution of components originating from animal species of high commercial value by those originating from species of less value, or even by components of plant origin such as soya.

This uncontrolled exploitation of biodiversity is even more pronounced in the fishing industry, where industrial processes such as the production of fillets or on-board freezing make it difficult, even impossible, to identify the species that are caught. In addition, this industrialization of fishing allows new species of fish to be caught, which are deep-sea fish.

The “interspecific” adulteration of food products is therefore at one and the same time a problem of economics, of public health and of preservation of the environment, which affects equally consumers, distributors, producers and the authorities responsible for food hygiene and fraud prevention.

It is therefore important to be able not only to determine the presence in food of biological materials originating from animal species, but also to identify the origin of these biological materials present in food.

In the agri-foodstuffs sector, the characterization of animal species makes use of biochemical techniques for the analysis of proteins (electrophoresis and immunoanalysis) or chemical techniques (principally chromatography). Thus, polyacrylamide gel electrophoresis under denaturing conditions was essentially developed for the analysis of cooked food (analysis of peptides or proteins denatured and coagulated by cooking (Patterson R. L. S., 1985, Biochemical identification of meat species, Elsevier ed)). This technique is now supplanted by pH gradient electrophoresis (isoelectric focalization or “IEF”), with much greater resolution. The fishing industry currently uses this method for the identification of species of fish (comparison of the electrophoretic profile of the muscle proteins of the studied species with an electrophoretic reference profile (Sotelo C. G. et al., 1993, Trends in Food Science and Technology, 4: 395-401)). However, isoelectric focalization is a technique that is difficult to use.

Although these techniques remain used for the identification of common species (in particular domesticated species) in food, the multiplication of the number of wild species affected (game, fish, crustaceans, shellfish, molluscs) means that the majority of these techniques are now at their limits.

Analysis of the genome by means of nucleic probes clearly represents a new alternative for the identification of species, one still little developed at the present time. In fact, the work carried out on ancient DNA (or fossil DNA) since the beginning of 1990s has demonstrated that DNA is a very stable molecule after death, despite the action of the time and of the environment (Brown et al., 1994, Bioessays, 16 (10): 719-26.). However, when it sur present in small quantities, in the forms of damaged and chemically modified molecules (Pääbo et al., 1989, Journal of Biology Chemistry, 264, 9709-9712). These characteristics are due essentially to the phenomena of hydrolysis and oxidation (Lindahl, 1993, Nature, 362, 709-715). Thanks to the polymerase chain reaction (“PCR” technique) which constitutes a tool of remarkable analytical power, it is possible to multiply a given DNA fragment in vitro in an almost exponential manner. By amplifying DNA of a food preparation which has undergone modifications such as cooking, salting, smoking, phenomena of hydrolysis and oxidation, it will be possible to identify each constituent of animal or plant origin. PCR was thus recently used for the characterization of cooked pigmeat (Meyer et al., 1995, Journal of AOAC International, 78, (6) 1542-1551)), sheep's meat or goat meat (Chikuni et al., 1994, Animal Science and Technology, 65 (6), 571-579) by amplification of sequences specific to the sought species. Similarly, the amplification of sequences specific to the Y chromosome allowed the determination of the sex of butchery carcasses of bovine and ovine origin (Cotinot C. et al., 1991, Genomics. 10 (3): 646-53, Apparao K. B. C., 1995, Meat Science, 39 (1) 123-126).

A subject of international application WO 98/50401 is a method for detecting the presence of biological materials of bovine origin in a sample of organic material. The method described in this reference makes use of PCR using suitable oligonucleotides, amplifying a part of the mitochondrial DNA control region. However, the method described in this reference allows only the detection of the presence or the absence of a single bovine species, namely the species Bos taurus, and thus does not allow the detection and identification of the presence of several species.

Fish belonging to the order of the gadiformes constitute the largest group of fish that are caught and sold, representing a little more than half of total fish catches. These fish are much used in the agri-foodstuffs sector (fillet, soup, terrine, pates, fish-based preparation, oils, flours . . . ) and are therefore very exposed to adulteration. The gadiformes belong to the class of the actinopterygii and form part of the teleostei or bony fish. They are divided into several families, such as the gadidae (whiting, cod, pollock, ling . . . ), the merluccidae (hake), the macrouridae (grenadier), the moridae (mora) and other families that are not industrially exploited.

From a technical point of view, studies have been conducted on the mitochondrial DNA (mtDNA) of the gadiformes, which is organized in the same way as for mammals. mtDNA is an excellent marker of species which is often used in phylogeny. In fact, depending on the species that is being studied, certain portions of mtDNA permit species to be differentiated one from the other, others have a finer power of resolution and allow different populations (geographical races, sub-species) to be distinguished: mtDNA replication control region, regions coding for cytochrome b or coding for the mitochondrial RNAs (RNA 12S or RNA 16S). The majority of the studies carried out on the mtDNA of the gadiformes analyse the genetic diversity of the species from a population point of view, comparing the sequences one with another or using microsatellite markers (Purcell et al., 1996. Molecular Marine Biology and Biotechnology, 5(3) 185-192; Ruzzante et al., 1998. Molecular Ecology. 7: 1663-1680; Lage and Kornfiels, 1999. Molecular Ecology 8: 1355-1357). Other studies are based on a precise region of the mtDNA, and calculate rates of divergence in order to study the evolution of one species relative to another (Carr et al., 1999. Canadian Journal of Zoology 77: 19-22). It is possible also to retrace their evolution and provide fresh data as regards the place of a species on a phylogenic tree (Morita., 1999. Molecular Phylogeny of Evolution 13 (3) 447-454). mtDNA has been sequenced in its entirety in a single species of gadidae: the Atlantic cod (Gadus morhua) (Johansen and Bakke, 1996. Molecular Marine Biology and Biotechnology 5(3) 203-214).

A study of the state of the art thus shows that work on the analysis of DNA, and in particular the mtDNA of certain species of gadiformes, has already been carried out. However, no document of the prior art describes a method allowing the detection of at least one species of fish (in particular of gadiformes) present in a sample of organic origin, and identification of the species present in said sample, by using the PCR technique using suitable oligonucleotides. No document of the prior art describes a specific, sensitive and reliable method of detecting and identifying degraded or non-degraded DNA, originating from one or more different species of gadiformes, in any sample of organic origin displaying very varied compositions that can contain fish. The applicant therefore endeavoured to develop a sensitive and reliable method that allows these shortcomings to be remedied.

One of the aims of the present invention is to provide a method for detecting the presence of biological materials originating from fish in a sample of organic material.

One of the other aims of the invention is to provide a method for identifying the genus, in particular of at least one species of fish present in a sample of organic material.

Another aim of the invention is to provide a method allowing species of fish which, though phylogenetically close, have different commercial values, to be distinguished one from the other.

Another aim of the invention is to provide a method of identifying the genus, in particular of at least one species of fish present in fresh or processed food (cooked, lyophilized, dried, pickled, appertized, pasteurized etc.).

A subject of the present invention is a method for detecting the presence of biological materials originating from gadiformes chosen from the group constituted by the gadidae, the merluccidae, the macrouridae and/or the moridae, in a sample of organic material, characterized in that the presence of mitochondrial DNA originating from gadiformes chosen from the group constituted by the gadidae, the merluccidae, the macrouridae and/or the moridae, is detected in said organic material by amplification of at least one sequence or fragment of mitochondrial DNA specific to the genome of the gadiformes chosen from the group constituted by the gadidae, the merluccidae, the macrouridae and/or the moridae, and contained in the mitochondrial DNA extracted from said sample, namely at least one sequence or fragment present in the genomes of the gadiformes chosen from the group constituted by the gadidae, the merluccidae, the macrouridae and/or the moridae, but absent from the genomes of the other animal genera, and in particular of the other animal species.

The present invention also has as a subject a method for detecting the presence of biological materials originating from gadiformes chosen from the group constituted by the gadidae, the merluccidae, the macrouridae and/or the moridae, in a sample of organic material, and for identifying the genus, in particular of at least one species of gadiformes chosen from the group constituted by the gadidae, the merluccidae, the macrouridae and/or the moridae, present in said sample, characterized in that the presence of mitochondrial DNA originating from gadiformes chosen from the group constituted by the gadidae, the merluccidae, the macrouridae and/or the moridae, is detected in said organic material by amplification of at least one sequence or fragment of mitochondrial DNA specific to the genome of the gadiformes chosen from the group constituted by the gadidae, the merluccidae, the macrouridae and/or the moridae, and contained in the mitochondrial DNA extracted from said sample, namely at least one sequence or fragment present in the genomes of the gadiformes chosen from the group constituted by the gadidae, the merluccidae, the macrouridae and/or the moridae, but absent from the genomes of the other animal genera, in particular of the other animal species, and in that at least one sequence or fragment of mitochondrial DNA specific to the genome of the gadiformes chosen from the group constituted by the gadidae, the merluccidae, the macrouridae and/or the moridae, thus amplified, is compared with other sequences of mitochondrial DNA of the genome of the gadiformes chosen from the group constituted by the gadidae, the merluccidae, the macrouridae and/or the moridae, said sequence of mitochondrial DNA or said fragment of mitochondrial DNA specific to the genome of the gadiformes chosen from the group constituted by the gadidae, the merluccidae, the macrouridae and/or the moridae, thus amplified, displaying at least approximately 50% identity, in particular approximately 60% identity with the other aforementioned sequences of mitochondrial DNA of the genome of the gadiformes chosen from the group constituted by the gadidae, the merluccidae, the macrouridae and/or the moridae.

By genus is meant an obligatory category to which every species must belong and which contains a species or a group of species (Wily, 1981).

By species is meant a population group really or partially capable of crossing, and which is reproductively isolated from the other groups having the same property. The animal species is divided into sub-species, races, varieties and strains; several neighbouring species form a genus, which for its part is a subdivision of the family.

By organic material is meant any solid or liquid material that can be presumed to have at least partially an organic origin.

The percentage identity relates to the result of the comparison of the nucleic acids of the DNA sequence that it is sought to identify with those of the known DNA sequences of the gadiformes chosen from the group constituted by the gadidae, the merluccidae, the macrouridae and/or the moridae.

Thus, when an amplified DNA sequence or fragment specific to the genome of the gadiformes displays at least approximately 50% identity, in particular approximately 60% with the other known DNA sequences of the genome of the gadiformes, it can be deduced from this that the sample of organic material to be analysed contains biological materials originating from gadiformes, such as the gadidae, the merluccidae, the macrouridae and/or the moridae.

When an amplified DNA sequence or fragment specific to the genome of the gadiformes displays less than approximately 50% identity with the other known DNA sequences of the genome of the gadiformes, it can be deduced from this that the sample of organic material to be analysed does not contain biological materials originating from gadiformes, such as the gadidae, the merluccidae, the macrouridae and/or the moridae.

According to an advantageous embodiment of the invention, the method allows the detection and optionally the identification of the presence of gadidae, in particular chosen from the group constituted by Gadus niorhua (common cod), Melanogranimus aeglefinus (haddock), Merlangius merlangus (whiting), Micromesistius poutassou (blue whiting), Pollachius virens (pollock), Pollachius pollachius (pollack), Trisopterus luscus (common pout), Trisopterus minutus capelanus (poor cod), Theragra chalcogramma (Alaskan pollock), Brosme brosme (tusk), Molva molva (ling) or Molva dypterygia dypterigia (blue ling).

According to another advantageous embodiment of the invention, the method allows the detection and optionally the identification of the presence of merluccidae, in particular chosen from the group constituted by Merluccius albidus (offshore hake), Merluccius australis (Southern hake), Merluccius bilinearis (silver hake), Merluccius capensis (shallow-water Cape hake), Merluccius gayi (Chilean hake), Merluccius hubbsi (Argentine hake), Merluccius merluccius (common hake), Merluccius paradoxus (deep-water Cape hake), Merluccius productus (North Pacific hake), Merluccius senegalensis (Senegalese hake), Steindachneria argentea (silver hake).

According to another advantageous embodiment of the invention, the method allows the detection and optionally the identification of the presence of macrouridae, in particular chosen from the group constituted by Abyssicola macrochir (abyssal grenadier), Albatrossia pectoralis (giant grenadier), Bathygadus macrops (grenadier sp.), Gadomus arcuatus (grenadier sp.), Coelorinchus argentatus (silver whiptail grenadier), Coryphaenoides acrolepis (Pacific grenadier), Coryphaenoides mexicanus (Mexican grenadier), Coryphaenoides rupestris (roundnose grenadier), Cynomacrurus pirei (dogtooth grenadier), Hymenocephalus italicus (Italian grenadier), Lepidorhynchus denticulatus (javelin grenadier), Macrourus berglax (grey grenadier), Malacocephalus laevis (bearded grenadier), Mataeocephalus acipenserinus (sturgeon grenadier), Nezumia aequalis (smooth grenadier), Sphagemacrurus hirundo (grayling grenadier), Trachonurus sulcatus (bristly grenadier), Trachyrincus helolepis (armourhead grenadier), Ventrifossa atherodon (arrowtooth grenadier).

According to another advantageous embodiment of the invention, the method allows the detection and optionally the identification of the presence of moridae, such as mora moro (common mora).

Advantageously, each of the amplified sequences or fragments of the genome of the gadiformes chosen from the group constituted by the gadidae, the merluccidae, the macrouridae and/or the moridae, is of mitochondrial origin.

The choice of a mitochondrial sequence is particularly advantageous as, in an animal cell there are approximately 100 to 1000 copies of mitochondrial DNA for one copy of nuclear DNA. In case of degradation of the DNA, the probability of detecting mitochondrial DNA is therefore much greater than the probability of detecting nuclear DNA. Mitochondrial DNA can therefore be more surely detected in organic materials in which the DNA is subject to various physical (temperature, pressure etc.), chemical or biochemical factors leading to its degradation.

According to an advantageous embodiment of the method of the invention, the DNA extracted from the sample of organic material is:

non-degraded DNA originating in particular from a fresh sample or,

degraded DNA, originating in particular from a sample that has been processed, in particular cooked, lyophilized, dried, pickled, appertized, pasteurized etc.

Preferably, the amplification of at least one DNA sequence or fragment specific to the genome of the gadiformes chosen from the group constituted by the gadidae, the merluccidae, the macrouridae and/or the moridae, is carried out by the polymerase chain amplification (PCR) method, comprising a repetition of the cycle of the following stages:

heating of the DNA extracted from the sample of organic material, so as to separate the DNA into two single-chain strands,

hybridization of oligonucleotide primers to the monocatenary DNA strands at an adequate temperature, and

elongation of the oligonucleotide primers by a polymerase at an adequate temperature, in order to obtain at least one amplified DNA sequence or fragment specific to the genome of the gadiformes chosen from the group constituted by the gadidae, the merluccidae, the macrouridae and/or the moridae.

In the above and in the following, each of the amplified DNA sequences or fragments obtained at the end of the polymerase chain reaction (PCR) using the primers of the invention, can also be called “amplified DNA fragment”, “DNA fragment”.

By “amplification product” is meant in the above and in the following the amplified DNA fragment or fragments or sequence or sequences obtained at the end of the polymerase chain reaction (PCR). The amplification product comprises several copies of different amplified DNA fragments or sequences when the sample of organic material to be analysed comprises a mixture of different DNA fragments originating from different species of gadiformes chosen from the group constituted by the gadidae, the merluccidae, the macrouridae and/or the moridae. The amplification product contains several copies of the same amplified DNA fragment or sequence when the sample of organic material to be analysed comprises several DNA fragments originating from the same species of gadiformes chosen from the group constituted by the gadidae, the merluccidae, the macrouridae and/or the moridae.

In the above and in the following, the oligonucleotide primers can also be called “oligonucleotides” or “primers”.

A subject of the invention is also a method for obtaining at least one DNA sequence or fragment originating from gadiformes, and in particular in gadidae such as those mentioned above, displaying a determined size and sequence, specific to the gadiformes, and in particular to the gadidae, from a sample of organic material, a method from which there is amplified, by polymerase chain reaction (PCR), at least one determined sequence of the genome of the gadiformes, and in particular of the gadidae, present in the genomes of the gadiformes, and in particular of the gadidae, but absent from the genomes of the other animal species.

A subject of the invention is also a method for obtaining at least one DNA sequence or fragment originating from gadiformes, and in particular in merluccidae such as those cited above, displaying a determined size and sequence, specific to the gadiformes, and in particular to the merluccidae, from a sample of organic material, a method from which there is amplified, by polymerase chain reaction (PCR), at least one determined sequence of the genome of the fishs, in particular of the gadiformes, and in particular of the merluccidae, present in the genomes of the gadiformes, and in particular of the merluccidae, but absent from the genomes of the other animal species.

In the above and in the following, the expression <<at least one DNA sequence or fragment>> means either that there are several copies of the same DNA fragment or sequence, or that there are several copies of different DNA fragments or sequences.

According to an advantageous embodiment of the method of the invention, each of the amplified sequences or fragments of the genome of the gadiformes chosen from the group constituted by the gadidae, the merluccidae, the macrouridae and/or the moridae, is situated in the central part of the gene coding for the cytochrome c oxidase of the mitochondrial DNA, delimited by the nucleotides situated in the vicinity of positions 6100 and 6601, and in particular in the vicinity of positions 6120 and 6590, and preferably in the vicinity of positions 6131 and 6580 of the gene coding for the cytochrome c oxidase of the mitochondrial DNA, said positions being defined according to Johansen and Bakke, 1996, Molecular Marine Biology and Biotechnology, 5(3) 203-214.

A subject of the invention is also the oligonucleotides chosen from those:

(1)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}1 (positions 6131 to 6154 according to Johansen and Bakke, 1996, Molecular Marine Biology and Biotechnology, 5(3) 203-214):

TRT AYC ARC AYY TRT TYT GRT TCT K

in which R is A or G, Y is C or T, K is G or T,

on condition that the following sequences are excluded:

CGG GAT CCT GTT CTG ATT CTT GAT TTC C
and
CGA CGG GAT CCC AAC ACC TGT TTC GAT CAT CGC GGC
AAC

or comprising the following sequence SEQ ID N^{o}2 (positions 6134 to 6154 according to Johansen and Bakke, 1996, Molecular Marine Biology and Biotechnology, 5(3) 203-214):

AYC ARC AYY TRT TYT GRT TCT

in which Y is C or T, R is A or G,

or constituted by the following sequence SEQ ID N^{o}3 (positions 6139 to 6153 according to Johansen and Bakke, 1996, Molecular Marine Biology and Biotechnology, 5(3) 203-214):

AYY TRT TYT GRT TCT

in which Y is C or T, R is A or G,

or those

(2)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}4 (positions 6244 to 6269 according to Johansen and Bakke, 1996, Molecular Marine Biology and Biotechnology, 5(3) 203-214):

TTY GGN YAY ATR GGN ATR GTN TGA GC

in which Y is C or T, N is A, C, G or T, R is A or G,

or comprising the following sequence SEQ ID N^{o}5 (positions 6247 to 6269 according to Johansen and Bakke, 1996, Molecular Marine Biology and Biotechnology, 5(3) 203-214):

GGN YAY ATR GGN ATR GTN TGA GC

in which N is A, C, G or T, Y is C or T, R is A or G,

or constituted by the following sequence SEQ ID N^{o}6 (positions 6253 to 6269 according to Johansen and Bakke, 1996, Molecular Marine Biology and Biotechnology, 5(3) 203-214):

RGG NAT RGT NTG AGC

in which R is A or G, N is A, C, G or T, or those

(3)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}7 (positions 6556 to 6580 according to Johansen and Bakke, 1996, Molecular Marine Biology and Biotechnology, 5(3) 203-214):

TAY GTW GTN GCN CAY TTY CAC TAC G

in which Y is C or T, W is A or T, N is A, C, G or T,

or comprising the following sequence SEQ ID N^{o}8 (positions 6556 to 6575 according to Johansen and Bakke, 1996, Molecular Marine Biology and Biotechnology, 5(3) 203-214):

TAY GTW GTN GCN CAY TTY CA

in which Y is C or T, W is A or T, N is A, C, G or T,

or constituted by the following sequence SEQ ID N^{o}9 (positions 6556 to 6570 according to Johansen and Bakke, 1996, Molecular Marine Biology and Biotechnology, 5(3) 203-214):

TAY GTW GTN GCN CAY

in which Y is C or T, W is A or T, N is A, C, G or T,

or those

(4)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}10 (positions 6131 to 6154 according to Johansen and Bakke, 1996, Molecular Marine Biology and Biotechnology, 5(3) 203-214):

TRT AYC ARC AYY TRT TYT GRT TCT K

in which R is A or G, Y is C or T, K is G or T,

or comprising the following sequence SEQ ID N^{o}11 (positions 6134 to 6154 according to Johansen and Bakke, 1996, Molecular Marine Biology and Biotechnology, 5(3) 203-214):

ACC AAC ACT TAT TCT GAT TCT

or constituted by the following sequence SEQ ID N^{o}12 (positions 6139 to 6154 according to Johansen and Bakke, 1996, Molecular Marine Biology and Biotechnology, 5(3) 203-214):

AYY AAC ACT TAT TCT

in which Y is C or T,

or those

(5)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}13 (positions 6277 to 6303 according to Johansen and Bakke, 1996, Molecular Marine Biology and Biotechnology, 5(3) 203-214):

CYA TYG GMC TYT YGG YTT TAT YGT V

in which Y is C or T, M is A or C, V is A, C or G,

or comprising the following sequence SEQ ID N^{o}14 (positions 6283 to 6303 according to Johansen and Bakke, 1996, Molecular Marine Biology and Biotechnology, 5(3) 203-214):

GGC CTC CTT GGC TTT ATT GTA

or constituted by the following sequence SEQ ID N^{o}15 (positions 6288 to 6303 according to Johansen and Bakke, 1996, Molecular Marine Biology and Biotechnology, 5(3) 203-214):

YTY GGY TTT ATT GTV

in which Y is C or T, V is A, C or G,

or those

(6)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}16 (positions 6496 to 6522 according to Johansen and Bakke, 1996, Molecular Marine Biology and Biotechnology, 5(3) 203-214):

GGM YTW ACA GGN ATY RTH YTR GCY AA

in which M is A or C, W is A or T, Y is C or T, N is A, C, G or T, R is A or G, H is A, C or T,

or comprising the following sequence SEQ ID N^{o}17 (positions 6496 to 6519 according to Johansen and Bakke, 1996, Molecular Marine Biology and Biotechnology, 5(3) 203-214):

GGC TTA ACA GGA ATT GTA CTA GCT

or constituted by the following sequence SEQ ID N^{o}18 (positions 6496 to 6510 according to Johansen and Bakke, 1996, Molecular Marine Biology and Biotechnology, 5(3) 203-214):

GGC TTA ACA GGA ATT

or those

(7)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}19 (positions 6195 to 6219 according to Johansen and Bakke, 1996, Molecular Marine Biology and Biotechnology, 5(3) 203-214):

CGG RAT AAT YTC YCA YAT YGT AGC C

in which R is A or G, Y is C or T,

or comprising the following sequence SEQ ID N^{o}20 (positions 6200 to 6219 according to Johansen and Bakke, 1996, Molecular Marine Biology and Biotechnology, 5(3) 203-214):

TAA TYT CYC AYA TYG TAG CC

in which Y is C or T,

or constituted by the following sequence SEQ ID N^{o}21 (positions 6205 to 6219 according to Johansen and Bakke, 1996, Molecular Marine Biology and Biotechnology, 5(3) 203-214):

TCY CAY ATY GTA GCC

in which Y is C or T,

or those

(8)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}22 (positions 6324 to 6348 according to Johansen and Bakke, 1996, Molecular Marine Biology and Biotechnology, 5(3) 203-214):

AGT BGG RAT RGA YGT DGA YAC MCG T

in which B is C, G or T, R is A or G, Y is C or T, D is A, G or T, M is A or C,

or comprising the following sequence SEQ ID N^{o}23 (positions 6329 to 6348 according to Johansen and Bakke, 1996, Molecular Marine Biology and Biotechnology, 5(3) 203-214):

GRA TRG AYG TDG AYA CMC GT

in which R is A or G, Y is C or T, D is A, G or T, M is A or C,

or constituted by the following sequence SEQ ID N^{o}24 (positions 6334 to 6348 according to Johansen and Bakke, 1996, Molecular Marine Biology and Biotechnology, 5(3) 203-214):

GAY GTD GAY ACM CGT

in which Y is C or T, D is A, G or T, M is A or C,

or those

(9)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}25 (positions 6498 to 6523 according to Johansen and Bakke, 1996, Molecular Marine Biology and Biotechnology, 5(3) 203-214):

ACT TAC AGG NAT YRT HCT RGC YAA YT

in which N is A, C, G or T, Y is C or T, R is A or G, H is A, C or T,

or comprising the following sequence SEQ ID N^{o}26 (positions 6498 to 6517 according to Johansen and Bakke, 1996, Molecular Marine Biology and Biotechnology, 5(3) 203-214):

ACT TAG AGG NAT YRT HCT RG

in which N is A, C, G or T, Y is C or T, R is A or G, H is A, C or T,

or constituted by the following sequence SEQ ID N^{o}27 (positions 6498 to 6512 according to Johansen and Bakke, 1996, Molecular Marine Biology and Biotechnology, 5(3) 203-214):

ACT TAC AGG NAT YRT

in which N is A, C, G or T, Y is C or T, R is A or G,

or those

(10)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}28 (positions 6399 to 6423 according to Johansen and Bakke, 1996, Molecular Marine Biology and Biotechnology, 5(3) 203-214):

AGT YTT YAG YTG AYT AGC AAC YYT V

in which Y is C or T, V is A, C or G,

or comprising the following sequence SEQ ID N^{o}29 (positions 6404 to 6423 according to Johansen and Bakke, 1996, Molecular Marine Biology and Biotechnology, 5(3) 203-214):

TTA GCT GAT TAG CAA CTT TA

or constituted by the following sequence SEQ ID N^{o}30 (positions 6409 to 6423 according to Johansen and Bakke, 1996, Molecular Marine Biology and Biotechnology, 5(3) 203-214):

TGA YTA GCA ACY YTV

in which Y is C or T, V is A, C or G,

or those

(11)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}31 (positions 6552 to 6577 according to Johansen and Bakke, 1996, Molecular Marine Biology and Biotechnology, 5(3) 203-214):

RTA YTA YGT ACT MGC YCA YTT YCA CT

in which R is A or G, Y is C or T, M is A or C,

or comprising the following sequence SEQ ID N^{o}32 (positions 6552 to 6572 according to Johansen and Bakke, 1996, Molecular Marine Biology and Biotechnology, 5(3) 203-214):

GTA TTA CGT AGT AGC CCA TT

or constituted by the following sequence SEQ ID N^{o}33 (positions 6552 to 6566 according to Johansen and Bakke, 1996, Molecular Marine Biology and Biotechnology, 5(3) 203-214):

RTA YTA YGT ACT MGC

in which R is A or G, Y is C or T, M is A or C,

or those

(12)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}34 (positions 6237 to 6261 according to Johansen and Bakke, 1996, Molecular Marine Biology and Biotechnology, 5(3) 203-214):

AGA RCC NTT YGG RYA YAT RGG HAT R

in which R is A or G, N is A, C, G or T, Y is C or T, H is A, C or T,

or comprising the following sequence SEQ ID N^{o}35 (positions 6242 to 6261 according to Johansen and Bakke, 1996, Molecular Marine Biology and Biotechnology, 5(3) 203-214):

CCT TTG GAT ATA TAG GCA TG

or constituted by the following sequence SEQ ID N^{o}36 (positions 6248 to 6261 according to Johansen and Bakke, 1996, Molecular Marine Biology and Biotechnology, 5(3) 203-214):

GGR YAY ATR GGH ATR

in which R is A or G, Y is C or T, H is A, C or T,

or those

(13)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}37 (positions 6381 to 6406 according to Johansen and Bakke, 1996, Molecular Marine Biology and Biotechnology, 5(3) 203-214):

YAT YCC RAC AGG YGT WAA AGT YTT YA

in which Y is C or T, R is A or G, W is A or T,

or comprising the following sequence SEQ ID N^{o}38 (positions 6381 to 6400 according to Johansen and Bakke, 1996, Molecular Marine Biology and Biotechnology, 5(3) 203-214):

TAT CCC AAC AGG TGT AAA AG

or constituted by the following sequence SEQ ID N^{o}39 (positions 6381 to 6395 according to Johansen and Bakke, 1996, Molecular Marine Biology and Biotechnology, 5(3) 203-214):

TAT YCC RAG AGG YGT

in which Y is C or T, R is A or G,

or those

(14)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}40 (positions 6267 to 6291 according to Johansen and Bakke, 1996, Molecular Marine Biology and Biotechnology, 5(3) 203-214):

AGC YAT RAT RGC YAT YGG MCT YCT Y

in which Y is C or T, R is A or G, M is A or C,

or comprising the following sequence SEQ ID N^{o}41 (positions 6272 to 6291 according to Johansen and Bakke, 1996, Molecular Marine Biology and Biotechnology, 5(3) 203-214):

TGA TGG CTA TTG GCC TCC TC

or constituted by the following sequence SEQ ID N^{o}42 (positions 6277 to 6291 according to Johansen and Bakke, 1996, Molecular Marine Biology and Biotechnology, 5(3) 203-214):

GCY ATY GGM CTY CTY

in which Y is C or T, M is A or C,

or those

(15)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}43 (positions 6451 to 6475 according to Johansen and Bakke, 1996, Molecular Marine Biology and Biotechnology, 5(3) 203-214):

HCC BMT MCT BTG RGC CCT V GG YTT YA

in which H is A, C or T, B is C, G or T, M is A or C, R is A or G, V is A, C or G, Y is C or T,

or comprising the following sequence SEQ ID N^{o}44 (positions 6451 to 6469 according to Johansen and Bakke, 1996, Molecular Marine Biology and Biotechnology, 5(3) 203-214):

CCC TCT ACT CTG AGC CCT AG

or constituted by the following sequence SEQ ID N^{o}45 (positions 6451 to 6464 according to Johansen and Bakke, 1996, Molecular Marine Biology and Biotechnology, 5(3) 203-214):

HCC BMT MCT BTG RGC

in which H is A, C or T, B is C, G or T, M is A or C, R is A or G,

or those

(16)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}46 (positions 6194 to 6219 according to Johansen and Bakke, 1996, Molecular Marine Biology and Biotechnology, 5(3) 203-214):

TSG RAT AAT YTC YCA YAT YGT AGC V

in which S is C or G, R is A or G, Y is C or T, V is A, C or G,

or comprising the following sequence SEQ ID N^{o}47 (positions 6200 to 6219 according to Johansen and Bakke, 1996, Molecular Marine Biology and Biotechnology, 5(3) 203-214):

TAA TTT CTC ACA TCG TAG CG

or constituted by the following sequence SEQ ID N^{o}48 (positions 6205 to 6219 according to Johansen and Bakke, 1996, Molecular Marine Biology and Biotechnology, 5(3) 203-214):

TCY CAY ATY GTA GCV

in which Y is C or T, V is A, C or G,

or those

(17)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}49 (positions 6342 to 6366 according to Johansen and Bakke, 1996, Molecular Marine Biology and Biotechnology, 5(3) 203-214):

YAC MCG WGC HTA CTT YAC ATC YGC A

in which Y is C or T, M is A or C, W is A or T, H is A, C or T

or comprising the following sequence SEQ ID N^{o}50 (positions 6342 to 6361 according to Johansen and Bakke, 1996, Molecular Marine Biology and Biotechnology, 5(3) 203-214):

TAC ACG TGC CTA CTT TAC AT

or constituted by the following sequence SEQ ID N^{o}51 (positions 6342 to 6356 according to Johansen and Bakke, 1996, Molecular Marine Biology and Biotechnology, 5(3) 203-214):

YAC MCG WGC HTA CTT

in which Y is C or T, M is A or C, W is A or T, H is A, C or T,

or those

(18)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}52 (positions 6152 to 6177 according to Johansen and Bakke, 1996, Molecular Marine Biology and Biotechnology, 5(3) 203-214):

TCT KCG GNC AYC CYG AAG THT AYA TH

in which K is G or T, N is A, C, G or T, Y is C or T, H is A, C or T,

or comprising the following sequence SEQ ID N^{o}53 (positions 6158 to 6177 according to Johansen and Bakke, 1996, Molecular Marine Biology and Biotechnology, 5(3) 203-214):

GAC ACC CCG AAG TAT ACA TA

or constituted by the following sequence SEQ ID N^{o}54 (positions 6163 to 6177 according to Johansen and Bakke, 1996, Molecular Marine Biology and Biotechnology, 5(3) 203-214):

CCY GAA GTH TAY ATH

in which Y is C or T, H is A, C or T,

or those

(19)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}55 (positions 6303 to 6328 according to Johansen and Bakke, 1996, Molecular Marine Biology and Biotechnology, 5(3) 203-214):

VTG RGC YCA YCA CAT RTT YAC AGT BG

in which V is A, C or G, R is A or G, Y is C or T, B is C, G or T,

or comprising the following sequence SEQ ID N^{o}56 (positions 6303 to 6322 according to Johansen and Bakke, 1996, Molecular Marine Biology and Biotechnology, 5(3) 203-214):

GTG AGC CCA TCA CAT GTT TA

or constituted by the following sequence SEQ ID N^{o}57 (positions 6303 to 6317 according to Johansen and Bakke, 1996, Molecular Marine Biology and Biotechnology, 5(3) 203-214):

VTG RGC YCA YCA CAT,

in which V is A, C or G, R is A or G, Y is C or T.

The oligonucleotides or primers as defined above allow the detection and optionally the identification of the DNA fragments originating from gadiformes chosen from the group constituted by the gadidae, the merluccidae, the macrouridae and/or the moridae.

A subject of the invention is also the pairs of primers constituted:

by any one of the oligonucleotides SEQ ID N^{o}1, SEQ ID N^{o}2, SEQ ID N^{o}3, SEQ ID N^{o}4, SEQ ID N^{o}5, SEQ ID N^{o}6, and any one of the oligonucleotides SEQ ID N^{o}7, SEQ ID N^{o}8, SEQ ID N^{o}9 as defined above or,

by any one of the oligonucleotides SEQ ID N^{o}10, SEQ ID N^{o}11, SEQ ID N^{o}12, SEQ ID N^{o}13, SEQ ID N^{o}14, SEQ ID N^{o}15, and any one of the oligonucleotides SEQ ID N^{o}16, SEQ ID N^{o}17, SEQ ID N^{o}18 as defined above or,

by any one of the oligonucleotides SEQ ID N^{o}19, SEQ ID N^{o}20, SEQ ID N^{o}21, SEQ ID N^{o}22, SEQ ID N^{o}23, SEQ ID N^{o}24, and any one of the oligonucleotides SEQ ID N^{o}25, SEQ ID N^{o}26, SEQ ID N^{o}27 as defined above or,

by any one of the oligonucleotides SEQ ID N^{o}28, SEQ ID N^{o}29, SEQ ID N^{o}30, and any one of the oligonucleotides SEQ ID N^{o}31, SEQ ID N^{o}32, SEQ ID N^{o}33 as defined above or,

by any one of the oligonucleotides SEQ ID N^{o}34, SEQ ID N^{o}35, SEQ ID N^{o}36, and any one of the oligonucleotides SEQ ID N^{o}37, SEQ ID N^{o}38, SEQ ID N^{o}39 as defined above or,

by any one of the oligonucleotides SEQ ID N^{o}40, SEQ ID N^{o}41, SEQ ID N^{o}42, and any one of the oligonucleotides SEQ ID N^{o}43, SEQ ID N^{o}44, SEQ ID N^{o}45 as defined above or,

by any one of the oligonucleotides SEQ ID N^{o}46, SEQ ID N^{o}47, SEQ ID N^{o}48, and any one of the oligonucleotides SEQ ID N^{o}49, SEQ ID N^{o}50, SEQ ID N^{o}51 as defined above or,

by any one of the oligonucleotides SEQ ID N^{o}52, SEQ ID N^{o}53, SEQ ID N^{o}54, and any one of the oligonucleotides SEQ ID N^{o}55, SEQ ID N^{o}56, SEQ ID N^{o}57 as defined above,

and advantageously constituted by the pair of oligonucleotides chosen from the following pairs:

(SEQ ID N^{o}2 and SEQ ID N^{o}8),

(SEQ ID N^{o}5 and SEQ ID N^{o}8),

(SEQ ID N^{o}11 and SEQ ID N^{o}17),

(SEQ ID N^{o}14 and SEQ ID N^{o}17),

(SEQ ID N^{o}20 and SEQ ID N^{o}26),

(SEQ ID N^{o}23 and SEQ ID N^{o}26),

(SEQ ID N^{o}29 and SEQ ID N^{o}32),

(SEQ ID N^{o}35 and SEQ ID N^{o}38),

(SEQ ID N^{o}41 and SEQ ID N^{o}44),

(SEQ ID N^{o}47 and SEQ ID N^{o}50),

(SEQ ID N^{o}53 and SEQ ID N^{o}56).

The pairs of primers (SEQ ID N^{o}2 and SEQ ID N^{o}8) and (SEQ ID N^{o}5 and SEQ ID N^{o}8) as defined above each allow the obtaining, by PCR reaction, of at least one DNA fragment originating from fish belonging to the order of the gadiformes, displaying a determined size and sequence, specific to the gadiformes.

The pairs of primers (SEQ ID N^{o}11 and SEQ ID N^{o}17) and (SEQ ID N^{o}14 and SEQ ID N^{o}17) as defined above each allow the obtaining, by PCR reaction, of at least one DNA fragment originating from fish belonging to the order of the gadiformes, and in particular to the family of the gadidae, displaying a determined size and sequence, specific to the gadidae.

The pairs of primers (SEQ ID N^{o}20 and SEQ ID N^{o}26) and (SEQ ID N^{o}23 and SEQ ID N^{o}26) as defined above each allow the obtaining, by PCR reaction, of at least one DNA fragment originating from fish belonging to the order of the gadiformes, and in particular to the family of the merluccidae, displaying a determined size and sequence, specific to the merluccidae.

The pair of primers (SEQ ID N^{o}29 and SEQ ID N^{o}32) as defined above allows the obtaining, by PCR reaction, of at least one DNA fragment originating from fish belonging to the order of the gadiformes, and in particular to the family of the gadidae, displaying a determined size and sequence, specific to the species Gadus morhua (Atlantic cod).

The pair of primers (SEQ ID N^{o}35 and SEQ ID N^{o}38) as defined above, allows the obtaining, by PCR reaction, of at least one DNA fragment originating from fish belonging to the order of the gadiformes, and in particular to the family of the gadidae, displaying a determined size and sequence, specific to the species Pollachius virens (pollock).

The pair of primers (SEQ ID N^{o}41 and SEQ ID N^{o}44) as defined above allows the obtaining, by PCR reaction, of at least one DNA fragment originating from fish belonging to the order of the gadiformes, and in particular to the family of the gadidae, displaying a determined size and sequence, specific to the species Theragra chalcogramma (Alaskan pollock).

The pair of primers (SEQ ID N^{o}47 and SEQ ID N^{o}50) as defined above allows the obtaining, by PCR reaction, of at least one DNA fragment originating from fish belonging to the order of the gadiformes, and in particular to the family of the gadidae, displaying a determined size and sequence, specific to the species Melanogrammus aeglefinus (haddock).

The pair of primers (SEQ ID N^{o}53 and SEQ ID N^{o}56) as defined above allows the obtaining, by PCR reaction, of at least one DNA fragment originating from fish belonging to the order of the gadiformes, and in particular to the family of the gadidae, displaying a determined size and sequence, specific to the species Merlangius merlangus (whiting).

A subject of the invention is also the DNA fragments as amplified at the end of the method as described above, comprising approximately 100 to approximately 500 base pairs.

A DNA fragment of the invention advantageously displays a sequence identity of at least 80%, preferably 90% and advantageously 95% with at least one of the sequences contained in:

the following SEQ ID N^{o}58:

| | | | |
|---|---|---|---|
| AYCARCAYYT | RTTCTGATTC | TKCGGNCAYC | CYGAAGTHTA |
| YATHCTNATY | YTMCCHGGMT | TCGGRATAAT | YTCYCAYATY |
| GTAGCVTAYT | AYTCAGGNAA | RMAAGARCCN | TTYGGRYAYA |
| TRGGHATRGT | NTGAGCYATR | ATRGCYATYG | GMCTYCTYGG |
| YTTTATYGTV | TGRGCYCAYC | ACATRTTYAC | AGTBGGRATR |
| GAYGTDGAYA | CMCGWGCHTA | CTTYACATCY | GCAACBATAA |
| TYATYGCYAT | YCCRACAGGY | GTWAAAGTYT | TYAGYTGAYT |
| AGCAACYYTV | CAYGGRGCCT | CARTTAARTG | RGAVACHCCB |
| MTMCTBTGRG | CCCTDGGYTT | YATYTTYCTM | TTYACMGTHG |
| GVGGMYTWAC | AGGNATYRTH | YTRGCYAAYT | CYTCYCTAGA |
| YATYGTDCTY | CAYGAYACRT | AYTAMGTAGT | MGCYCAYTTY |
| CA | | | |

in which Y is C or T, R is A or G, K is G or T, N is A, C, G or T, H is A, C or T, M is A or C, V is A, C or G, B is C, G or T, D is A, G or T, W is A or T,

said sequence SEQ ID N^{o} 58 comprising 442 base pairs,

or the following SEQ ID N^{o}59:

| | | | |
|---|---|---|---|
| GGRYAYATRG | GHATRGTNTG | AGCYATRATR | GCYATYGGMC |
| TYCTYGGYTT | TATYGTVTGR | GCYCAYCACA | TRTTYACAGT |
| BGGRATRGAY | GTDGAYACMC | GWGCHTACTT | YACATCYGCA |
| ACBATAATYA | TYGCYATYCC | RACAGGYGTW | AAAGTYTTYA |
| GYTGAYTAGC | ACYYTVCAYG | GRGGCTCART | TAARTGRGAV |
| ACHCCBMTMC | TBTGRGCCCT | DGGYTTYATY | TTYCTMTTYA |
| CMGTHGGVGG | MYTWACAGGN | ATYRTHYTRG | CYAAYTCYTC |
| YCTAGAYATY | GTDCTYCAYG | AYACRTAYTA | MGTAGTMGCY |
| CAYTTYCA | | | |

in which R is A or G, Y is C or T, K is G or T, N is A, C, G or T, H is A, C or T, M is A or C, V is A, C or G, B is C, G or T, D is A, G or T, W is A or T,

said sequence SEQ ID N^{o} 59 comprising 328 base pairs,

or the following SEQ ID N^{o}60:

AYCARCAYYT RTTCTGATTC TKCGGNCAYC CYGAAGTHTA
YATHCTNATY YTMCCHGGMT TCGGRATAAT YTCYCAYATY
GTAGCVTAYT AYTCAGGNAA RMAAGARCCN TTYGGRYAYA
TRGGHATRGT NTGAGCYATR ATRGCYATYG GMCTYCTYGG
YTTTATYGTV TGRGCYCAYC ACATRTTYAC AGTBGGRATR
GAYGTDGAYA CMCGWGCHTA CTTYACATCY GCAACBATAA
TYATYGCYAT YCCRACAGGY GTWAAAGTYT TYAGYTGAYT
AGCAACYYTV CAYGGRGGCT CARTTAARTG RGAVACHCCB
MTMCTBTGRG CCCTDGGYTT YATYTTYCTM TTYACMGTHG
GVGGMYTWAC AGGNATYRTH YTRGCY

in which Y is C or T, R is A or G, K is G or T, N is A, C, G or T, H is A, C or T, M is A or C, V is A, C or G, B is C, G or T, D is A, G or T, W is A or T,

said sequence SEQ ID N^{o}60 comprising 386 base pairs,

or the following SEQ ID N^{o}61:

GGMCTYCTYG GYTTTATYGT VTGRGCYCAY CACATRTTYA
CAGTBGGRAT RGAYGTDGAY ACMCGWGCHT ACTTYACATC
YGCAACBATA ATYATYGCYA TYCCRACAGG YGTWAAAGTY
TTYAGYTGAY TAGCAACYYT VCAYGGRGGC TCARTTAART
GRGAVACHCC BMTMCTBTGR GCCCTDGGYT TYATYTTYCT
MTTYACMGTH GGVGGMYTWA CAGGNATYRT HYTRGCY

in which Y is C or T, R is A or G, K is G or T, N is A, C, G or T, H is A, C or T, M is A or C, V is A, C or G, B is C, G or T, D is A, G or T, W is A or T,

said sequence SEQ ID N^{o} 61 comprising 237 base pairs,

or the following SEQ ID N^{o}62:

TAATYTCYCA YATYGTAGCV TAYTAYTCAG GNAARMAAGA
RCCNTTYGGR YAYATRGGHA TRGTNTGAGC YATRATRGCY
ATYGGMCTYC TYGGYTTTAT YGTVTGRGCY CAYCACATRT
TYACAGTBGG RATRGAYGTD GAYACMCGWG CHTACTTYAC
ATCYGCAACB ATAATYATYG CYATYCCRAC AGGYGTWAAA
GTYTTYAGYT GAYTAGCAAC YYTVCAYGGR GGCTCARTTA
ARTGRGAVAC HCCBMTMCTB TGRGCCCTDG GYTTYATYTT
YCTMTTYACM GTHGGVGGMY TWACAGGNAT YRTHYTRG

in which Y is C or T, R is A or G, K is G or T, N is A, C, G or T, H is A, C or T, M is A or C, V is A, C or G, B is C, G or T, D is A, G or T, W is A or T,

said sequence SEQ ID N^{o} 62 comprising 318 base pairs,

or the following SEQ ID N^{o}63:

GRATRGAYGT DGAYACMCGW GCHTACTTYA CATCYGCAAC
BATAATYATY GCYATYCCRA CAGGYGTWAA AGTYTTYAGY
TGAYTAGCAA CYYTVCAYGG RGGCTCARTT AARTGRGAVA
CHCCBMTMCT BTGRGCCCTD GGYTTYATYT TYCTMTTYAC
MGTHGGVGGM YTWACAGGNA TYRTHYTRG

in which R is A or G, Y is C or T, D is A, G or T, M is A or C, W is A or T, B is C, G or T, V is A, C or G, H is A, C or T, N is A, C, G or T,

said sequence SEQ ID N^{o}63 comprising 189 base pairs,

or the following SEQ ID N^{o}64:

TYAGYTGAYT AGCAACYYTV CAYGGRGGCT CARTTAARTG
RGAVACHCCB MTMCTBTGRG CCCTDGGYTT YATYTTYCTM
TTYACMGTHG GVGGMYTWAC AGGNATYRTH YTRGCYAAYT
CYTCYCTAGA YATYGTDCTY CAYGAYACRT AYTAMGTAGT
MGCYCAYT

in which Y is C or T, V is A, C or G, R is A or G, B is C, G or T, H is A, C or T, M is A or C, D is A, G or T, N is A, C, G or T,

said sequence SEQ ID N^{o}64 comprising 168 base pairs,

or the following SEQ ID N^{o}65:

CNTTYGGRYA YATRGGHATR GTNTGAGCYA TRATRGCYAT
YGGMCTYCTY GGYTTTATYG TVTGRGCYCA YCACATRTTY
ACAGTBGGRA TRGAYGTDGA YACMCGWGCH TACTTYACAT
CYGCAACBAT AATYATYGCY ATYCCRACAG GYGTWAAAG

in which N is A, C, G or T, R is A or G, Y is C or T, H is A, C or T, M is A or C, V is A, C or G, B is C, G or T, D is A, G or T, W is A or T,

said sequence comprising 159 base pairs,

or the following sequence SEQ ID N^{o}66:

TRATRGCYAT YGGMCTYCTY GGYTTTATYG TVTGRGCYCA
YCACATRTTY ACAGTBGGRA TRGAYGTDGA YACMCGWGCH
TACTTYACAT CYGCAACBAT AATYATYGCY ATYCCRACAG
GYGTWAAAGT YTTYAGYTGA YTAGCAACYY TVCAYGGRGG
CTCARTTAAR TGRGAVACHC CBMTMCTBTG RGCCCTDG

in which R is A or G, Y is C or T, M is A or C, V is A, C or G, B is C, G or T, D is A, G or T, W is A or T, H is A, C or T,

said SEQ ID N^{o}66 comprising 198 base pairs,

the following SEQ ID N^{o}67:

TAATYTCYCA YATYGTAGCV TAYTAYTCAG GNAARMAAGA
RCCNTTYGGR YAYATRGGHA TRGTNTGAGC YATRATRGCY
ATYGGMCTYC TYGGYTTTAT YGTVTGRGCY CAYCACATRT
TYACACTBGG RATRGAYGTD GAYACMCGWG CHTACTTYAC
AT

in which Y is C or T, V is A, C or G, N is A, C, G or T, R is A or G, M is A or C, H is A, C or T, B is C, G or T, D is A, G or T, W is A or T,

said sequence SEQ ID N^{o}67 comprising 162 base pairs,

or the following SEQ ID N^{o}68:

GNCAYCCYGA AGTHTAYATH CTNATYYTMC CHGGMTTCGG
RATAATYTCY CAYATYGTAG CVTAYTAYTC AGGNAARMAA
GARCCNTTYG GRYAYATRGG HATRGTNTGA GCYATRATRG
CYATYGGMCT YCTYGGYTTT ATYGTVTGRG CYCAYCACAT
RTTYA

in which N is A, C, G or T, Y is C or T, H is A, C or T, M is A or C, R is A or G, V is A, C or G,

said sequence SEQ ID N^{o} 68 comprising 165 base pairs.

The DNA fragments SEQ ID N^{o}58 and SEQ ID N^{o}59 as defined above are specific to fish belonging to the order of the gadiformes. The DNA fragments SEQ ID N^{o}60 and SEQ ID N^{o}61 as defined above are specific to fish belonging to the order of the gadiformes, and more particularly to fish belonging to the family of the gadidae. The DNA fragments SEQ ID N^{o}62 and SEQ ID N^{o}63 as defined above are specific to fish belonging to the order of the gadiformes, and more particularly to fish belonging to the family of the merluccidae.

The DNA fragment SEQ ID N^{o}64 as defined above is specific to fish belonging to the order of the gadiformes, more particularly to fish belonging to the family of the gadidae, and more particularly the species Gadus morhua (Atlantic cod). The DNA fragment SEQ ID N^{o}65 as defined above is specific to fish belonging to the order of the gadiformes, more particularly to fish belonging to the family of the gadidae, and more particularly to the species Pollachius virens (pollock). The DNA fragment SEQ ID N^{o}66 as defined above is specific to fish belonging to the order of the gadiformes, more particularly to fish belonging to the family of the gadidae, and more particularly to the species Theragra chalcogramma (Alaskan pollock). The DNA fragment SEQ ID N^{o}67 as defined above is specific to fish belonging to the order of the gadiformes, more particularly to fish belonging to the family of the gadidae, and more particularly to the species Melanogrammus aeglefinus (haddock). The DNA fragment SEQ ID N^{o}68 as defined above is specific to fish belonging to the order of the gadiformes, more particularly to fish belonging to the family of the gadidae, and more particularly to the species Merlangius merlangus (whiting).

According to an advantageous embodiment of the method of the invention, the obtained amplified DNA fragment(s) contained in the amplification product is (are) identified:

by sequencing of at least one amplified DNA fragment contained in the amplification product, and in particular of one amplified DNA fragment or,

directly, by visualization of the presence of the amplification product by gel electrophoresis.

According to an advantageous embodiment of the invention, the method of identification by sequencing of at least one obtained amplified DNA fragment allows the identification of the fish belonging to the order of the gadiformes, in particular chosen from the group constituted by the gadidae, the merluccidae, the macrouridae and/or the moridae.

The method of direct identification by simple visualization of the presence of the amplification product allows the identification of the species of fish belonging to the order of the gadiformes, and more particularly of the species of fish belonging to the family of the gadidae.

Each of the two methods of identification is described in more detail below.

1) The method of identification by sequencing of at least one obtained amplified DNA fragment is called <<global strategy>> in the following, as it allows the detection and identification of the presence or the absence of all the existing gadiformes. By way of guidance, a list of gadiformes that are able to be detected and identified by the method of the invention is given in the table 1 below. This list is not exhaustive, however.

The primers used in the global strategy in order to effect the amplification by the PCR method of at least one DNA sequence or fragment specific to the genome of the gadiformes, possibly present in a sample of organic material to be analysed, are the 27 primers listed below and as defined above:

SEQ ID N^{o}1, SEQ ID N^{o}2, SEQ ID N^{o}3, SEQ ID N^{o}4, SEQ ID N^{o}5, SEQ ID N^{o}6, SEQ ID N^{o}7, SEQ ID N^{o}8, SEQ ID N^{o}9, SEQ ID N^{o}10, SEQ ID N^{o}11, SEQ ID N^{o}12, SEQ ID N^{o}13, SEQ ID N^{o}14, SEQ ID N^{o}15, SEQ ID N^{o}16, SEQ ID N^{o}17, SEQ ID N^{o}18, SEQ ID N^{o}19, SEQ ID N^{o}20, SEQ ID N^{o}21, SEQ ID N^{o}22, SEQ ID N^{o}23, SEQ ID N^{o}24, SEQ ID N^{o}25, SEQ ID N^{o}26 and SEQ ID N^{o}27,

and more particularly the 9 primers SEQ ID N^{o}2, SEQ ID N^{o}5, SEQ ID N^{o}8, SEQ ID N^{o}11, SEQ ID N^{o}14, SEQ ID N^{o}17, SEQ ID N^{o}20, SEQ ID N^{o}23 and SEQ ID N^{o}26.

The pairs of primers advantageously used are as defined above.

According to a particularly advantageous implementation of the present invention, a part of the stages of hybridization of the cycles constituting the amplification reaction is carried out at a temperature of approximately 50° C. to approximately 58° C. Moreover, the applicant found that a temperature of 55° C. was particularly suitable for obtaining a specific amplification. Such a mode of implementation allows a greater specificity of amplification to be achieved.

It will be noted on this point that the applicant has solved a certain number of technical problems for the implementation of this method. First of all, the choice of primers constituted a real problem, as it was necessary to select on the one hand sequences common to the different species of gadiformes but not to other species of fish, and on the other hand hybridizing in stable manner, in very variable physico-chemical conditions, representative of the great variability of the organic materials likely to contain biological materials originating from gadiformes. The temperatures of the stages of separation of the strands and of elongation are advantageously respectively approximately 94° C. and approximately 72° C.

The method described above is specific to the gadiformes but global between all the species of gadiformes as it does not give an amplification reaction detectable in the presence of DNA of an origin other than the gadiformes.

According to an advantageous embodiment of the method of the invention (global strategy), the use of the following pairs of oligonucleotide primers:

(SEQ ID N^{o}2 and SEQ ID N^{o}8),

(SEQ ID N^{o}5 and SEQ ID N^{o}8),

(SEQ ID N^{o}11 and SEQ ID N^{o}17),

(SEQ ID N^{o}14 and SEQ ID N^{o}17),

(SEQ ID N^{o}20 and SEQ ID N^{o}26) and

(SEQ ID N^{o}23 and SEQ ID N^{o}26),

allows respectively the obtaining of:

a DNA fragment SEQ ID N^{o}58 as defined above,

a DNA fragment SEQ ID N^{o}59 as defined above,

a DNA fragment SEQ ID N^{o}60 as defined above,

a DNA fragment SEQ ID N^{o}61 as defined above,

a DNA fragment SEQ ID N^{o}62 as defined above and,

a DNA fragment SEQ ID N^{o}63 as defined above.

The DNA fragments described above, obtained at the end of the PCR reaction, can be detected even when a substantial fraction of the DNA is degraded, namely after action by physical, chemical and/or biochemical factors, and during various transformations of the samples of organic material to be analysed.

The described method displays a great simplicity of interpretation, because of the production of a single and unique amplification product, specific to the DNA of gadiformes and which therefore is not found in the DNA amplifications products of other orders. The uniqueness of the amplification product obtained at the end of the PCR reaction represents another advantage of the present invention, that of allowing of a substantial sensitivity to be achieved and greatly facilitating the interpretation of the results. The method according to the present invention offers a large number of advantages compared with already known identification techniques.

The amplification product can be shown by any method known to a person skilled in the art, and in particular by simple agarose gel electrophoresis. The reading of the migration profiles of the amplification product obtained with the method of the invention therefore simply consists of determining the presence of a single and unique migration band in an electrophoresis gel. In the absence of such a band, it can be considered that there are no detectable traces of DNA of gadiformes. The presence of a band signifies on the other hand that the DNA of gadiformes is present in the sample, and therefore that the sample in question contains biological material based on gadiformes.

When the obtained amplification product comprises several copies of the same amplified DNA fragment or sequence, this latter can then be sequenced in order to determine its nucleotide sequence. The comparison of the nucleotide sequence obtained with all the known nucleotide sequences of the gadiformes allows the determination of the species of gadiforme present in the sample of organic material, and thus the differentiation of the species from one another.

When the obtained amplification product comprises several copies of different amplified DNA fragments or sequences, the sequencing of each of the different amplified DNA fragments will be preceded by a cloning method. Thus, according to an advantageous embodiment of the method of the invention, the sequencing of each of the different amplified DNA fragments is preceded by a cloning method when the sample of organic material comprises a mixture of different DNA fragments originating from different species of gadiformes chosen from the group constituted by the gadidae, the merluccidae, the macrouridae and/or the moridae, said cloning method permitting the separation from said mixture of the different DNA fragments originating from the different species of fish.

According to an advantageous embodiment of the method of the invention, the presence of DNA originating from gadiformes in a sample of organic material is detected by amplification of at least one DNA sequence specific to the genome of the gadiformes using any one of the oligonucleotides SEQ ID N^{o}1 SEQ ID N^{o}2, SEQ ID N^{o}3, SEQ ID N^{o}4, SEQ ID N^{o}5, SEQ ID N^{o}6, and any one of the oligonucleotides SEQ ID N^{o}7, SEQ ID N^{o}8, SEQ ID N^{o}9 as defined above,

and advantageously using the pair of oligonucleotides (SEQ ID N^{o}2 and SEQ ID N^{o}8) or the pair of oligonucleotides (SEQ ID N^{o}5 and SEQ ID N^{o}8), in order to obtain respectively at least one of the DNA sequences contained in SEQ ID N^{o}58 or in SEQ ID N^{o}59 as defined above, said sequences being specific to the genome of the gadiformes,

and in that at least one species of gadiforme present in said sample of organic material is identified by sequencing of at least one of the DNA sequences contained in SEQ ID N^{o}58 or in SEQ ID N^{o}59, said sequencing being optionally preceded by a cloning method when the amplification product obtained at the end of the PCR reaction comprises a mixture of different DNA fragments or sequences originating from different species of gadiformes.

Thus, the pairs of oligonucleotides (SEQ ID N^{o}2 and SEQ ID N^{o}8) or (SEQ ID N^{o}5 and SEQ ID N^{o}8) allow, by amplification followed by a sequencing, the detection and identification of all the existing gadiformes, whatever the family concerned (gadidae, merluccidae, macrouridae etc.).

According to another advantageous embodiment of the method of the invention, the presence of DNA originating from gadidae in a sample of organic material is detected by amplification of at least one DNA sequence specific to the genome of the gadidae using any one of the oligonucleotides SEQ ID N^{o}10, SEQ ID N^{o}11, SEQ ID N^{o}12, SEQ ID N^{o}13, SEQ ID N^{o}14, SEQ ID N^{o}15, and any one of the oligonucleotides SEQ ID N^{o}16, SEQ ID N^{o}17, SEQ ID N^{o}18 as defined above,

and advantageously using the pair of oligonucleotides (SEQ ID N^{o}11 and SEQ ID N^{o}17) or the pair of oligonucleotides (SEQ ID N^{o}14 and SEQ ID N^{o}17), in order to obtain respectively at least one of the DNA sequences contained in SEQ ID N^{o}60 or in SEQ ID N^{o}61 as defined above, said sequences being specific to the genome of the gadidae,

and in that at least one species of gadidae present in said sample of organic material is identified by sequencing of at least one of the DNA sequences contained in SEQ ID N^{o}60 or in SEQ ID N^{o}61, said sequencing being optionally preceded by a cloning method when the amplification product obtained at the end of the PCR reaction comprises a mixture of different DNA fragments or sequences originating from different species of gadidae.

Thus the pairs of oligonucleotides (SEQ ID N^{o}11 and SEQ ID N^{o}17) or (SEQ ID N^{o}14 and SEQ ID N^{o}17) allow, by amplification followed by a sequencing, the detection and identification of all the existing gadidae, whatever the species or genus considered, but do not allow the detection and identification of the gadiformes not belonging to the family of the gadidae.

According to another advantageous embodiment of the method of the invention, the presence of DNA originating from merluccidae in a sample of organic material is detected by amplification of at least one DNA sequence specific to the genome of the merluccidae using any one of the oligonucleotides SEQ ID N^{o}19, SEQ ID N^{o}20, SEQ ID N^{o}21, SEQ ID N^{o}22, SEQ ID N^{o}23, SEQ ID N^{o}24, and any one of the oligonucleotides SEQ ID N^{o}25, SEQ ID N^{o}26, SEQ ID N^{o}27 as defined above,

and advantageously using the pair of oligonucleotides (SEQ ID N^{o}20 and SEQ ID N^{o}26) or the pair of oligonucleotides (SEQ ID N^{o}23 and SEQ ID N^{o}26), in order to obtain respectively at least one of the DNA sequences contained in SEQ ID N^{o}62 or in SEQ ID N^{o}63 as defined above, said sequences being specific to the genome of the merluccidae,

and in that at least one species of merluccidae present in said sample of organic material is identified by sequencing of at least one of the DNA sequences contained in SEQ ID N^{o}62 or in SEQ ID N^{o}63, said sequencing being optionally preceded by a cloning method when the amplification product obtained at the end of the PCR reaction comprises a mixture of different DNA fragments or sequences originating from different species of merluccidae.

Thus, the pairs of oligonucleotides (SEQ ID N^{o}20 and SEQ ID N^{o}26) and (SEQ ID N^{o}23 and SEQ ID N^{o}26) allow, by amplification followed by a sequencing, the detection and identification of all the existing merluccidae, whatever the species or genus considered, but do not allow the detection and identification of the gadiformes not belonging to the family of the merluccidae.

2) The method of direct identification by simple visualization of the presence of the amplification product using a gel electrophoresis is called <<specific strategy >> in the following, as it allows the detection and direct identification of very specific species of gadidae. In the <<specific strategy >>, the stage of detection and of identification is a simultaneous stage.

In fact, this method, unlike the method described above (“global strategy”), does not require an additional identification stage at the end of the detection stage (the detection of the presence of biological materials originating from gadiformes being possible by the obtaining of an amplification product), and thus allows the detection and identification directly after amplification of certain very particular species of fish, which in the present case are species which are very familiar through being much used in food or other preparations. The species of fish that can be simultaneously detected and identified according to the method of the present invention are more particularly the following five species of gadidae:

Gadus morhua (common or Atlantic cod),

Pollachius virens (pollock),

Theragra chalcogramma (Alaskan pollock),

Melanogrammus aeglefinus (haddock) and

Merlangius merlangus (whiting).

The amplification of the DNA is carried out by the polymerase chain amplification method (PCR) as described previously.

The specific strategy, allowing the simultaneous detection and identification of the presence of very specific species of gadidae in a sample of organic material, is described in more detail below.

(a) Specific strategy allowing the identification of Gadus morhua (common or Atlantic cod).

The primers used in the specific strategy in order to effect the amplification by the PCR method of a DNA sequence originating from gadidae, and more particularly Gadus morhua, possibly present in a sample of organic material to be analysed, are the 6 primers listed below and as defined above:

SEQ ID N^{o}28, SEQ ID N^{o}29, SEQ ID N^{o}30, SEQ ID N^{o}31, SEQ ID N^{o}32, SEQ ID N^{o}33, and more particularly the 2 primers SEQ ID N^{o}29 and SEQ ID N^{o}32.

According to an advantageous embodiment of the method of the invention (“specific strategy”), the use of the pair of oligonucleotides (SEQ ID N^{o}29 and SEQ ID N^{o}32) allows an amplification product containing a DNA fragment SEQ ID N^{o}64 as defined above to be obtained. The presence of said amplification product is detected by simple visualization by agarose gel electrophoresis, and thus allows the simultaneous detection and identification of the presence of Gadus morhua (common or Atlantic cod).

The expression “amplification product containing a DNA fragment” used in paragraph 2) above and hereafter must be understood to mean “amplification product containing several copies of the same DNA fragment or sequence”.

(b) Specific strategy allowing the identification of Pollachius virens (pollock).

The primers used in the specific strategy in order to effect the amplification by the PCR method of a DNA sequence originating from gadidae, and more particularly in Pollachius virens, possibly present in a sample of organic material to be analysed, are the 6 primers listed below and as defined above:

SEQ ID N^{o}34, SEQ ID N^{o}35, SEQ ID N^{o}36, SEQ ID N^{o}37, SEQ ID N^{o}38, SEQ ID N^{o}39, and more particularly the 2 primers SEQ ID N^{o}35 and SEQ ID N^{o}38.

According to an advantageous embodiment of the method of the invention, the use of the pair of oligonucleotides (SEQ ID N^{o}35 and SEQ ID N^{o}38), allows an amplification product containing a DNA fragment SEQ ID N^{o}65 as defined above to be obtained. The presence of said amplification product is detected by simple visualization by agarose gel electrophoresis, and thus allows the simultaneous detection and identification of the presence of Pollachius virens (pollock).

(c) Specific strategy allowing the identification of Theragra chalcogramma (Alaskan pollock).

The primers used in the specific strategy in order to effect the amplification by the PCR method of a DNA sequence originating from gadidae, and more particularly in Theragra chalcogramma, possibly present in a sample of organic material to be analysed, are the 6 primers listed below and as defined above:

SEQ ID N^{o}40, SEQ ID N^{o}41, SEQ ID N^{o}42, SEQ ID N^{o}43, SEQ ID N^{o}44, SEQ ID N^{o}45, and more particularly the 2 primers SEQ ID N^{o}41 and SEQ ID N^{o}44.

According to an advantageous embodiment of the method of the invention, the use of the pair of oligonucleotides (SEQ ID N^{o}41 and SEQ ID N^{o}44) allows an amplification product containing a DNA fragment SEQ ID N^{o}66 as defined above to be obtained. The presence of said amplification product is detected by simple visualization by agarose gel electrophoresis, and thus allows the simultaneous detection and identification of the presence of Theragra chalcogramma (Alaskan pollock).

According to a particularly advantageous implementation of the present invention, in the specific strategy allowing the identification of respectively Gadus morhua (common or Atlantic cod), Pollachius virens (pollock) and Theragra chalcogramma (Alaskan pollock), a part of the stages of hybridization of the cycles constituting the amplification reaction is carried out at a temperature of 60° C. which is particularly suitable for achieving a specific amplification. Such an implementation allows a greater specificity of amplification to be achieved.

(d) Specific strategy allowing the identification of Melanogrammus aeglefinus (haddock).

The primers used in the specific strategy in order to effect the amplification by the PCR method of a DNA sequence originating from gadidae, and more particularly in Melanogrammus aeglefinus, possibly present in a sample of organic material to be analysed, are the 6 primers listed below and as defined above:

SEQ ID N^{o}46, SEQ ID N^{o}47, SEQ ID N^{o}48, SEQ ID N^{o}49, SEQ ID N^{o}50, SEQ ID N^{o}51, and more particularly the 2 primers SEQ ID N^{o}47 and SEQ ID N^{o}50.

According to an advantageous embodiment of the method of the invention, the use of the pair of oligonucleotides (SEQ ID N^{o}47 and SEQ ID N^{o}50) allows an amplification product containing a DNA fragment SEQ ID N^{o}67 as defined above to be obtained. The presence of said amplification product is detected by simple visualization by agarose gel electrophoresis, and thus allows the simultaneous detection and identification of the presence of Melanogrammus aeglefinus (haddock).

(e) Specific strategy allowing the identification of Merlangius merlangus (whiting).

The primers used in the specific strategy in order to effect the amplification by the PCR method of a DNA sequence originating from gadidae, and more particularly in Merlangius merlangus, possibly present in a sample of organic material to be analysed, are the 6 primers listed below and as defined above:

SEQ ID N^{o}52, SEQ ID N^{o}53, SEQ ID N^{o}54, SEQ ID N^{o}55, SEQ ID N^{o}56, SEQ ID N^{o}57, and more particularly the 2 primers SEQ ID N^{o}53 and SEQ ID N^{o}56.

According to an advantageous embodiment of the method of the invention, the use of the pair of oligonucleotides (SEQ ID N^{o}53 and SEQ ID N^{o}56), allows respectively an amplification product containing a DNA fragment SEQ ID N^{o}68 as defined above to be obtained. The presence of said amplification product is detected by simple visualization by gel agarose electrophoresis, and thus allows the simultaneous detection and identification of the presence of Merlangius merlangus (whiting).

According to a particularly advantageous implementation of the present invention, in the specific strategy allowing the identification of respectively Melanogrammus aeglefinus (haddock) and Merlangius merlangus (whiting), a part of the stages of hybridization of the cycles constituting the amplification reaction is carried out at a temperature of 65° C. which is particularly suitable for obtaining a specific amplification. Such an implementation allows a greater specificity of amplification to be achieved.

According to an advantageous embodiment of the method of the invention, the presence of DNA originating from gadiformes in a sample of organic material, in particular in gadidae chosen from the group constituted by the species Gadus morhua, Pollachius virens, Theragra chalcogramma, Melanogrammus aeglefinus and Merlangius merlangus, is detected and each of the aforementioned species is identified by amplification of at least one DNA sequence specific to the genome of each of the aforementioned species of gadidae, with the help respectively:

of any one of the oligonucleotides SEQ ID N^{o}28, SEQ ID N^{o}29, SEQ ID N^{o}30, and any one of the oligonucleotides SEQ ID N^{o}31, SEQ ID N^{o}32, SEQ ID N^{o}33 as defined above or,

of any one of the oligonucleotides SEQ ID N^{o}34, SEQ ID N^{o}35, SEQ ID N^{o}36, and any one of the oligonucleotides SEQ ID N^{o}37, SEQ ID N^{o}38, SEQ ID N^{o}39 as defined above or,

of any one of the oligonucleotides SEQ ID N^{o}40, SEQ ID N^{o}41, SEQ ID N^{o}42, and any one of the oligonucleotides SEQ ID N^{o}43, SEQ ID N^{o}44, SEQ ID N^{o}45 as defined above or,

of any one of the oligonucleotides SEQ ID N^{o}46, SEQ ID N^{o}47, SEQ ID N^{o}48, and any one of the oligonucleotides SEQ ID N^{o}49, SEQ ID N^{o}50, SEQ ID N^{o}51 as defined above or,

of any one of the oligonucleotides SEQ ID N^{o}52, SEQ ID N^{o}53, SEQ ID N^{o}54, and any one of the oligonucleotides SEQ ID N^{o}55, SEQ ID N^{o}56, SEQ ID N^{o}57 as defined above,

and advantageously with the help respectively:

of the pair of oligonucleotides (SEQ ID N^{o}29 and SEQ ID N^{o}32) or,

of the pair of oligonucleotides (SEQ ID N^{o}35 and SEQ ID N^{o}38) or,

of the pair of oligonucleotides (SEQ ID N^{o}41 and SEQ ID N^{o}44) or,

of the pair of oligonucleotides (SEQ ID N^{o}47 and SEQ ID N^{o}50) or,

of the pair of oligonucleotides (SEQ ID N^{o}53 and SEQ ID N^{o}56), in order to obtain respectively at least one of the DNA sequences contained in:

SEQ ID N^{o}64 specific to the genome of Gadus morhua (Atlantic cod),

SEQ ID N^{o}65 specific to the genome of Pollachius virens (pollock),

SEQ ID N^{o}66 specific to the genome of Theragra chalcogramma (Alaskan pollock),

SEQ ID N^{o}67 specific to the genome of Melanogrammus aeglefinus (haddock),

SEQ ID N^{o}68 specific to the genome of Merlangius merlangus (whiting),

said SEQ ID N^{o}64, SEQ ID N^{o}65, SEQ ID N^{o}66, SEQ ID N^{o}67 and SEQ ID N^{o}68 being as defined above.

Thus the pairs of oligonucleotides defined above allow the detection and direct identification by amplification of a very particular species of gadidae.

According to an advantageous embodiment of the method of the invention (global strategy), when the experimenter suspects the presence of several different species of gadiformes in the sample of organic material to be analysed, the cloning method will be carried out directly at the end of the stage of amplification by PCR reaction of the DNA extracted from the sample. At the end of the cloning method the sequencing of each of the different DNA fragments or sequences corresponding respectively to one and the same species of gadiformes will be carried out.

According to another advantageous embodiment of the method of the invention (global strategy), when the experimenter suspects the presence of a single species of gadiforme in the sample of organic material to be analysed, the sequencing will be carried out directly at the end of the stage of amplification by PCR reaction of the DNA extracted from the sample. However if numerous indeterminations appear on the profile obtained at the end of the sequencing, this means that the sample of organic material to be analysed contains at least two different species of gadiformes. In this case, it will be necessary to proceed with the cloning in order to then carry out the sequencing of each of the different DNA fragments or sequences corresponding respectively to one and the same species of gadiformes.

According to another advantageous embodiment of the method of the invention (specific strategy), when the experimenter suspects that several different species of gadiformes are present in the sample of organic material to be analysed, he will successively carry out several amplifications by PCR reaction, using each of the appropriate pairs of oligonucleotide primers, in order to detect and to identify in a single stage the presence of each of the species of gadiformes which he suspects are present in the sample of organic material to be analysed (such as Gadus morhua, and/or Pollachius virens, and/or Theragra chalcogramma, and/or Melanogrammus aeglefinus, and/or Merlangius merlangus).

According to another advantageous embodiment of the method of the invention (specific strategy), when the experimenter suspects that a single species of gadiforme is present in the sample of organic material to be analysed, he will carry out a single amplification by PCR reaction, using the appropriate pair of oligonucleotide primers, in order to detect and to identify in a single stage the presence of the species of gadiforme which he suspects is present in the sample of organic material to be analysed (such as Gadus morhua or Pollachius virens or Theragra chalcogramma or Melanogrammus aeglefinus, or Merlangius merlangus).

A subject of the invention is also the use of nucleotide sequences chosen from the oligonucleotide primers as defined above or following sequences: CGG GAT CCT GTT CTG ATT CTT GAT TTC C or CGA CGG GAT CCC AAC ACC TGT TTC GAT CAT CGC GGC AAC, or DNA fragments as defined above, for the implementation of a method for detecting the presence of biological materials originating from gadiformes chosen from the group constituted by the gadidae, the merluccidae, the macrouridae and/or the moridae, and optionally of identifying at least one species of gadiformes chosen from the group constituted by the gadidae, the merluccidae, the macrouridae and/or the moridae present, in a sample of organic material likely to contain such biological materials, and in particular in fresh or indeed processed products such as agri-foodstuffs products, in particular fillet, soup, terrine, pâtés, fat, flour, fish-based preparations etc.

### DESCRIPTION OF THE FIGURES

FIG. 1 represents an agarose gel coloured with ethidium bromide. A marker measuring 20 kilo base pairs (Kpb) (M) is deposited on the gel. The DNAs are extracted from different samples of organic materials such as pollock fillet (well 1), salted cod (well 2), a cooked dish with Alaskan pollock (well 3), fish rillettes (well 4) and fish soup (well 5), and are deposited on the gel.

FIGS. 2-a and 2-b represent an agarose gel coloured with ethidium bromide. A marker measuring 100 bp (M) is deposited on the gel. The oligonucleotides of the present invention were tested beforehand on reference DNAs (DNA of gadiformes).

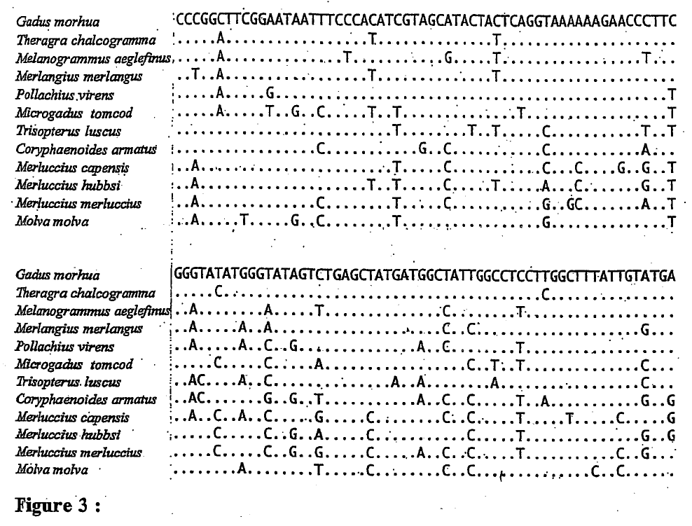

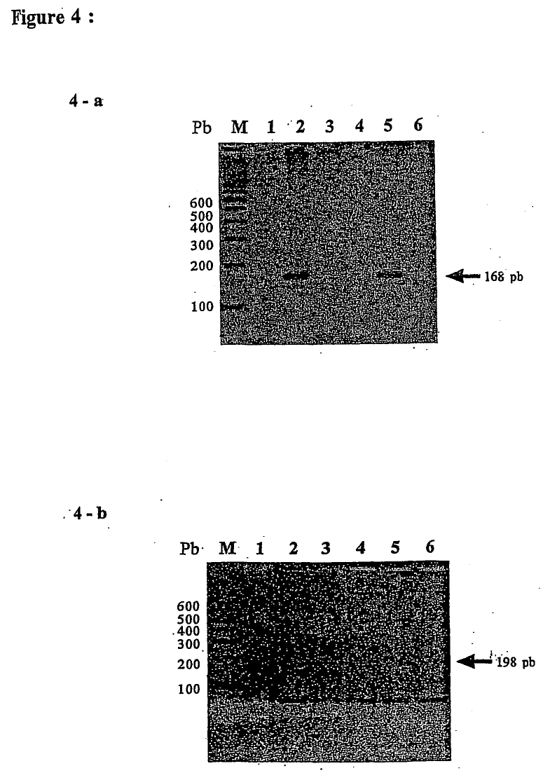

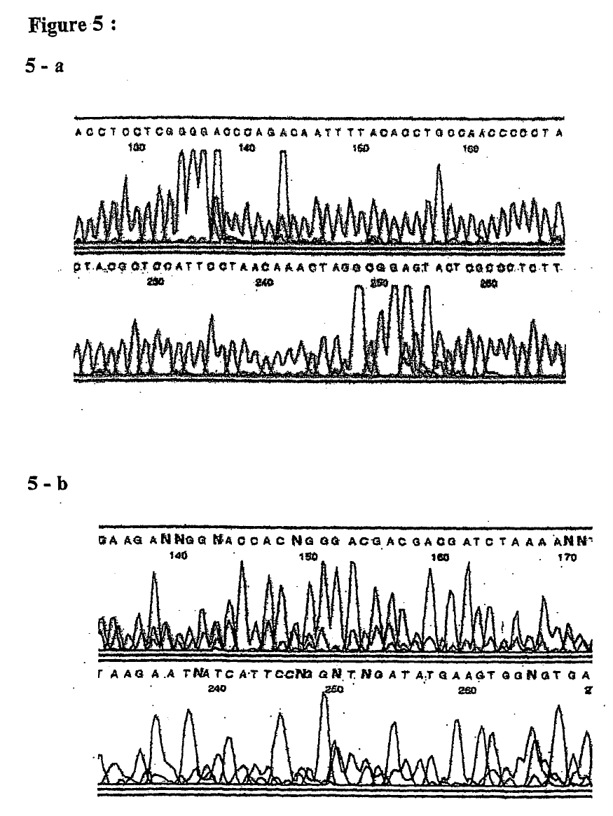

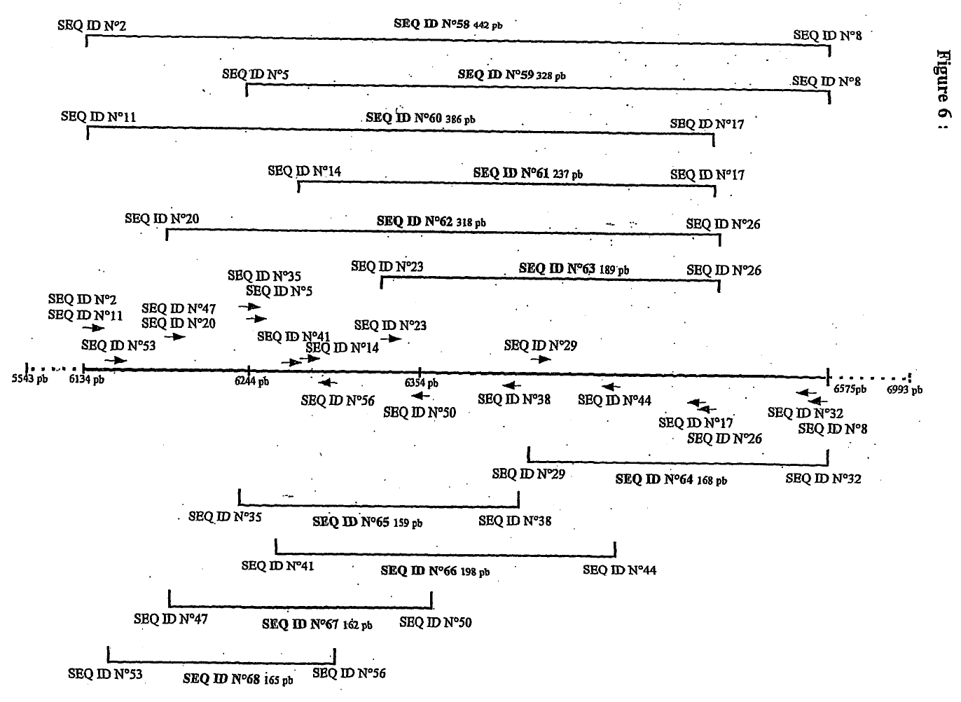

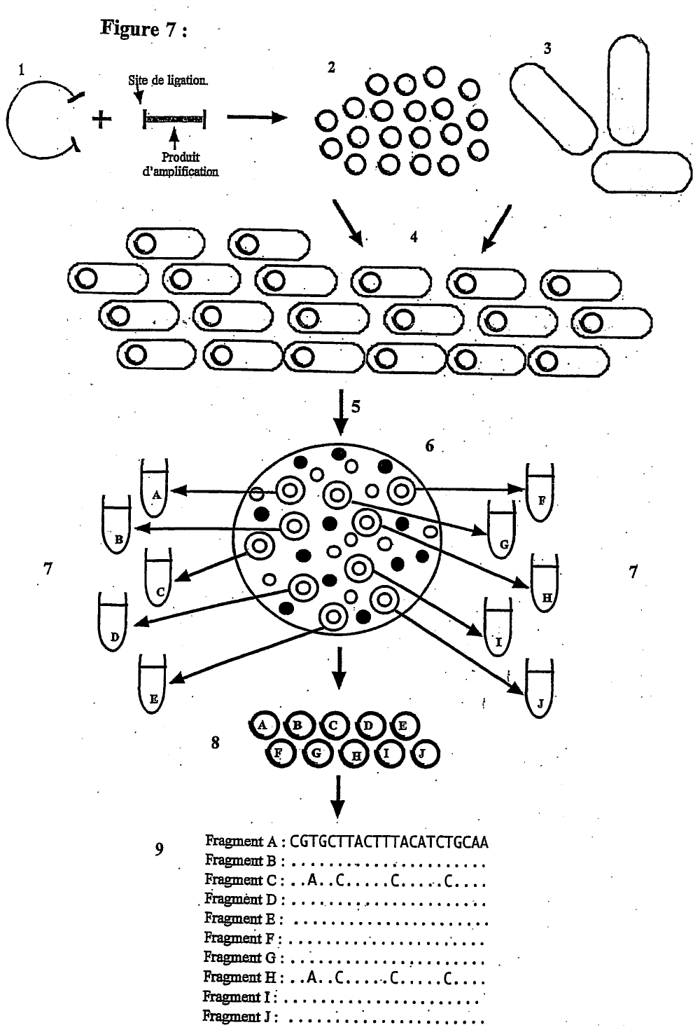

In FIG. 2-a, the DNAS of gadiformes (wells 1, 2) were amplified using the primers SEQ ID N^{o}2 and SEQ ID N^{o}8. The amplification product obtained, containing at least one fragment SEQ ID N^{o}58, migrates forming a band of 442 base pairs, specific to the genome of the gadiformes. The DNAs of gadiformes (wells 3, 4) were amplified using the primers SEQ ID N^{o}5 and SEQ ID N^{o}8. The amplification product obtained, containing at least one fragment SEQ ID N^{o}59, migrates forming a band of 328 base pairs, specific to the genome of the gadiformes. Well 5 is a negative amplification control.

In FIG. 2-b the DNAs of gadiformes (wells 2, 3) were amplified using the primers SEQ ID N^{o}20 and SEQ ID N^{o}26. The amplification product obtained, containing at least one fragment SEQ ID N^{o}62, migrates forming a band of 318 base pairs, specific to the genome of the merluccidae. Well 1 is a negative amplification control. The DNAs of gadiformes (wells 5, 6) were amplified using the primers SEQ ID N^{o}23 and SEQ ID N^{o}26. The amplification product obtained, containing at least one fragment SEQ ID N^{o}63, migrates forming a band of 189 base pairs, specific to the genome of the merluccidae. Well 4 is a negative amplification control.

FIG. 3 represents alignments of nucleotide sequences of 120 base pairs (120 bp) of the gene coding for the cytochrome c oxidase of the mitochondrial DNA of different species of fish, in particular gadiformes chosen from the group constituted by the gadidae, the merluccidae, the macrouridae and/or the moridae. The sequences obtained after sequencing are aligned and compared with the already known sequences. The sequences of the different species of fish represented are respectively the following (from top to bottom of the figure): Gadus morhua (common or Atlantic cod), Theragra chalcogramma (Alaskan pollock), Melanogrammus aeglefinus (haddock), Merlangius merlangus (whiting), Pollachius virens (pollock), Microgadus tomcod (Atlantic tomcod), Trisopterus luscus (common pout), Coryphaenoides armatus (grenadier), Merluccius capensis (shallow-water Cape hake), Merluccius hubbsi (Argentine hake), Merluccius merluccius (common hake) and Molva molva (ling). The dots represent the bases preserved with those of Gadus morhua (reference sequence).

FIGS. 4-a and 4-b represent an agarose gel coloured with ethidium bromide on which is deposited a marker measuring 100 bp (M). These figures represent more particularly tests carried out according to the specific strategy, on different food preparations.

In FIG. 4-a, the DNAs extracted from different food preparations, fillet (well 1), brandade (well 2), cooked dish (well 3), soup (well 4) and rillettes (well 5), were amplified using the primers SEQ ID N^{o}29 and SEQ ID N^{o}32. The amplification product obtained, containing a fragment SEQ ID N^{o}64, migrates forming a band of 168 base pairs, specific to Gadus morhua. Well 6 is a negative amplification control. The absence of amplification product for wells 1, 3 and 4 indicates that there is no Gadus morhua in the food preparations tested. On the other hand, the presence of an amplification product in wells 2 and 5 indicates the presence of Gadus morhua in the food preparations tested.

In FIG. 4-b, the DNAs extracted from various food preparations, fillet (well 1), brandade (well 2), cooked dish (well 3), soup (well 4) and rillettes (well 5) were amplified using the primers SEQ ID N^{o}41 and SEQ ID N^{o}44. The amplification product obtained, containing a fragment SEQ ID N^{o}66, migrates forming a band of 198 base pairs, specific to Theragra chalcogramma. Well 6 is a negative amplification control. The absence of amplification product for wells 2, 4 and 5 indicates that there is no Theragra chalcogramma in the food preparations tested. On the other hand the presence of an amplification product in wells 1 and 3 indicates the presence of Theragra chalcogramma in the food preparations tested.

FIGS. 5-a and 5-b represent profiles of crude sequences obtained according to the global strategy.

FIG. 5-a represents more particularly the profile obtained after extraction of the DNA of a food preparation of the cooked dish type, and amplification using the primers SEQ ID N^{o}2 and SEQ ID N^{o}8. The amplification product obtained, containing at least one fragment SEQ ID N^{o}58, forms a band of 442 base pairs, specific to the genome of the gadiformes. The sequence obtained at the end of the sequencing is clear and no indetermination appears on the profile. It can therefore be deduced from this that a single species of gadiforme is present in the food preparation. In the present case, analysis of the sequence reveals the presence of Gadus morhua in the food preparation.

FIG. 5-b represents more particularly the profile obtained after extraction of the DNA of a food preparation of the cooked dish type, and amplification using the primers SEQ ID N^{o}2 and SEQ ID N^{o}8. The amplification product obtained, containing at least one fragment SEQ ID N^{o}58, forms a band of 442 base pairs, specific to the genome of the gadiformes. The sequence obtained at the end of the sequencing presents numerous indeterminations (N) which appear on the profile. It can therefore be deduced from this that the food preparation contains a mixture of at least two species of gadiformes. In the present case it will be necessary, in order to determine precisely the species of gadiformes present in the food preparation, to carry out a cloning of the amplification product obtained, containing at least one fragment SEQ ID N^{o}58, said cloning allowing separation of the different species of gadiformes present in the food preparation. At the end of the cloning, it will then be possible to proceed with the sequencing of each of the fragments, and thus to determine the different species present in the food preparation.

FIG. 6 represents the positions, on the gene coding for the cytochrome c oxidase of the mitochondrial DNA, said positions being defined by Johansen and Bakke, 1996, Molecular Marine Biology and Biotechnology, 5(3) 203-214:

of the oligonucleotide primers of the invention, namely SEQ ID N^{o}1, SEQ ID N^{o}2, SEQ ID N^{o}3, SEQ ID N^{o}4, SEQ ID N^{o}5, SEQ ID N^{o}6, SEQ ID N^{o}7, SEQ ID N^{o}8, SEQ ID N^{o}9, SEQ ID N^{o}10, SEQ ID N^{o}11, SEQ ID N^{o}12, SEQ ID N^{o}13, SEQ ID N^{o}14, SEQ ID N^{o}15, SEQ ID N^{o}16, SEQ ID N^{o}17, SEQ ID N^{o}18, SEQ ID N^{o}19, SEQ ID N^{o}20, SEQ ID N^{o}21, SEQ ID N^{o}22, SEQ ID N^{o}23, SEQ ID N^{o}24, SEQ ID N^{o}25, SEQ ID N^{o}26, SEQ ID N^{o}27, SEQ ID N^{o}28, SEQ ID N^{o}29, SEQ ID N^{o}30, SEQ ID N^{o}31, SEQ ID N^{o}32, SEQ ID N^{o}33, SEQ ID N^{o}34, SEQ ID N^{o}35, SEQ ID N^{o}36, SEQ ID N^{o}37, SEQ ID N^{o}38, SEQ ID N^{o}39, SEQ ID N^{o}40, SEQ ID N^{o}41, SEQ ID N^{o}42, SEQ ID N^{o}43, SEQ ID N^{o}44, SEQ ID N^{o}45, SEQ ID N^{o}46, SEQ ID N^{o}47, SEQ ID N^{o}48, SEQ ID N^{o}49, SEQ ID N^{o}50, SEQ ID N^{o}51, SEQ ID N^{o}52, SEQ ID N^{o}53, SEQ ID N^{o}54, SEQ ID N^{o}55, SEQ ID N^{o}56, SEQ ID N^{o}57 and,

of the DNA fragments amplified with the help of the oligonucleotide primers of the invention, namely SEQ ID N^{o}58, SEQ ID N^{o}59, SEQ ID N^{o}60, SEQ ID N^{o}61, SEQ ID N^{o}62, SEQ ID N^{o}63, SEQ ID N^{o}64, SEQ ID N^{o}65, SEQ ID N^{o}66, SEQ ID N^{o}67 and SEQ ID N^{o}68.

FIG. 7 represents the cloning method used within the framework of the invention in order to separate the different DNA fragments originating from different species of fish in the same mixture.

Part 1 of FIG. 7 represents the pCR®2.1—TOPO plasmid in linear form. The amplification product of the invention, which contains several copies of different DNA fragments, is placed in contact with the pCR® 2.1—TOPO plasmid, using a ligase enzyme.

Part 2 represents a part of each of the DNA fragments (initially contained in the amplification product) ligated in a pCR®2.1—TOPO plasmid. A plasmid corresponds to each DNA fragment.

Part 3 represents the Escherichia coli (E. coli) bacterial cells.

In part 4, the E. coli bacteria are transformed by introduction of the plasmids represented in part 2. A plasmid corresponds to each E. coli bacterium.

Part 5 (symbolized by an arrow) represents the spread of the E. coli bacteria on a medium containing an antibiotic (for example ampicillin), in order to select the bacteria that have incorporated a plasmid.

Part 6 represents a dish of gelose on which blue colonies (symbolized by ●) and white colonies (symbolized by ◯) have grown. The blue colonies are characteristic of the bacterial cells in which the DNA fragments are not ligated to the plasmid, whereas the white colonies are characteristic of the bacterial cells in which the DNA fragments are ligated with the plasmid.

Part 7 represents the individual sampling of the colonies of white bacteria (for example 10 bacterial colonies selected at random and symbolized by the letters A to J) which are individually cultured, as a plasmid, and therefore a specific DNA fragment (symbolized respectively by the letters A, B, C, D, E, F, G, H, I and J) corresponds to each bacterial colony.

In part 8, the bacteria were eliminated in order to recover the plasmids and their DNA fragments (A to J). Sufficient plasmid DNA is thus obtained in order to sequence it.

Part 9 represents the result of the sequencing of the different DNA fragments A to J, carried out using two primers specific to the plasmid which frame the DNA fragment that it is wished to sequence. From the ten fragments sequenced, two different types of sequences are obtained. Thus, the analysed mixture comprises two different species of gadiformes, in this case Gadus morhua (fragments A, B, D, E, F, G, I and J), and Merluccius hubbsi (fragments C and H).

Table 1 below represents a list of gadiformes that can be detected and identified according to the method of the invention. This list is not exhaustive, however; thus, species of gadiformes other than those specifically named in table 1 below can be detected and identified according to the method of the invention.

TABLE 1

| Families | Genus | Species | Common name |
|---|---|---|---|
| Bregmacerotidae | Bregmaceros | Bregmaceros sp. | unicorn cod |
| Gadidae | Arctogadus | Arctogadus glacialis | artic cod |
| | Boreogadus | Boreogadus saida | polar cod |
| | Brosme | Brosme brosme | tusk |
| | Ciliata | Ciliata mustela | five-bearded rockling |
| | Eleginus | Eleginus gracilis | saffron cod |
| | | Eleginus navaga | wachna cod |
| | Enchelyopus | Enchelyopus cimbrius | four-bearded rockling |
| | Gadiculus | Gadiculus argenteus | silver pollock |
| | Gadus | Gadus macrocephalus | Pacific cod |
| | | Gadus morhua | common cod |
| | | Gadus ogac | Greenland cod |
| | Gaidropsarus | Gaidropsarus vulgaris | common rockling |
| | | Gaidropsarus mediterraneus | three-bearded rockling |
| | Lota | lota lota | burbot |
| | Melanogrammus | Melanogrammus aeglefinus | haddock |
| | Merlangius | Merlangius merlangus | whiting |
| | Microgadus | Microgadus proximus | tomcod |
| | | Microgadus tomcod | Atlantic tomcod |
| | Micromesistius | Micromesistius poutassou | blue whiting |
| | | Micromesistius australis | Southern whiting |
| | Molva | Molva dypterygia | blue ling |
| | | Molva molva | ling |
| | Phycis | Phycis blennoides | greater forkbeard |
| | | Phycis chesteri | longfin hake |
| | | Phycis phycis | forkbeard |
| | Pollachius | Pollachius pollachius | pollack |
| | | Pollachius virens | pollock |
| | Raniceps | Raniceps raninus | tadpole fish |
| | Theragra | Theragra chalcogramma | Alaskan pollock |
| | | Theragra finnmarchica | Norwegian pollock |
| | Trisopterus | Trisopterus esmarkii | Norway pout |
| | | Trisopterus luscus | common pout |
| | | Trisopterus capelanus | poor cod |
| | | Trisopterus minutus minutus | little poor cod |
| | Urophycis | Urophycis brasiliensis | Brazilian codling |
| | | Urophycis chuss | red hake |
| | | Urophycis floridana | Southern hake |
| | | Urophycis regia | spotted codling |
| | | Urophycis tenuis | white hake |
| Macrouridae | Abyssicola | Abyssicola macrochir | abyssal grenadier |
| | Albatrossia | Albatrossia pectoralis | giant grenadier |
| | Bathygadus | Bathygadus macrops | Grenadier sp. |
| | Gadomus | Gadomus arcuatus | Grenadier sp. |
| | Coelorinchus | Coelorinchus argentatus | silver whiptail |
| | | | grenadier |
| | Coryphaenoides | Coryphaenoides acrolepis | Pacific grenadier |
| | | Coryphaenoides mexicanus | Mexican grenadier |
| | | Coryphaenoides rupestris | roundnose grenadier |
| | Cynomacrurus | Cynomacrurus pirei | dogtooth grenadier |
| | Hymenocephalus | Hymenocephalus italicus | Italian grenadier |
| | Lepidorhynchus | Lepidorhynchus denticulatus | javelin grenadier |
| | Macrous | Macrourus berglax | grey grenadier |
| | Malacocephalus | Malacocephalus laevis | bearded grenadier |
| | Mataeocephalus | Mataeocephalus | sturgeon grenadier |
| | | acipenserinus | |
| | Nezumia | Nezumia aequalis | smooth grenadier |
| | Sphagemacrurus | Sphagemacrurus hirundo | grayling grenadier |
| | Trachonurus | Trachonurus sulcatus | bristly grenadier |
| | Trachyrincus | Trachyrincus halolepis | armourhead grenadier |
| | Ventrifossa | Ventrifossa atherodon | arrowtooth grenadier |
| Merluccidae | Merluccius | Merluccius albidus | offshore hake |
| | | Merluccius australis | Southern hake |
| | | Merluccius bilinearis | silver hake |
| | | Merluccius capensis | shallow-water hake |
| | | Merluccius gayi | Chilean hake |
| | | Merluccius hubbsi | Argentine hake |
| | | Merluccius merluccius | common hake |
| | | Merluccius paradoxus | deep-water Cape hake |
| | | Merluccius productus | Pacific hake |
| | | Merluccius senegalensis | Senegalese hake |
| | Steindachneria | Steindachneria argentea | silver hake |
| Moridae | Antimora | Antimora microlepsis | purple antimora |
| | Auchenoceros | Auchenoceros punctatus | ahuru |
| | Mora | Mora moro | common mora |

The examples below illustrate the invention. They in no way limit it.

### EXAMPLE 1

### Extraction of DNA

In order to be able to develop a method of detection by gene amplification of biological materials originating from gadiformes in products used in agri-foodstuff production, and in all the other fields using organic material, the experiments described in this first example were carried out with various types of samples representing potential sources of the presence of biological materials originating from fish.

1) Extraction of DNA by the Phenol/Chloroform Method

This method is suitable for all the types of samples likely to contain organic material, such as fillet, soup, terrine, pate, fat, flour, fish-based preparations etc.

This method makes use of the technique described in the references HÄNNI et al., 1990, C.R. Acad. Sci. Paris., 310, 365-370 and HÄNNI et al., 1995, Nucl. Acids Res., 23, 881-882, concerning the extraction of DNA from bones and teeth.

A quantity of approximately 1 to 2 g of sample of organic material is incubated for two hours at 37° C. in 400 μl of lysis buffer of the following composition:

STE 1× (NaCl 100 mM, Tris 10 mM at pH 7.4, EDTA (ethyl enediaminetetraacetic acid) 1 mM),

SDS 2%,

proteinase K at 0.5 mg/ml.

The proteinase K allows the proteins to be degraded and the nucleic acids released. The lysate is then extracted twice with a volume of phenol/chloroform (1/1). This is centrifuged for 15 minutes at 1000 g, the organic phase is eliminated, which allows the protein part of the lysate to be disposed of. The DNA is precipitated by 1/10 of volume of 2M sodium acetate then by 2.5 volumes of isopropanol and centrifuged for 30 minutes at 10 000 g. The DNA is taken up in water: a DNA extract is thus obtained. The volume of water is a function of the quantity of recovered DNA.

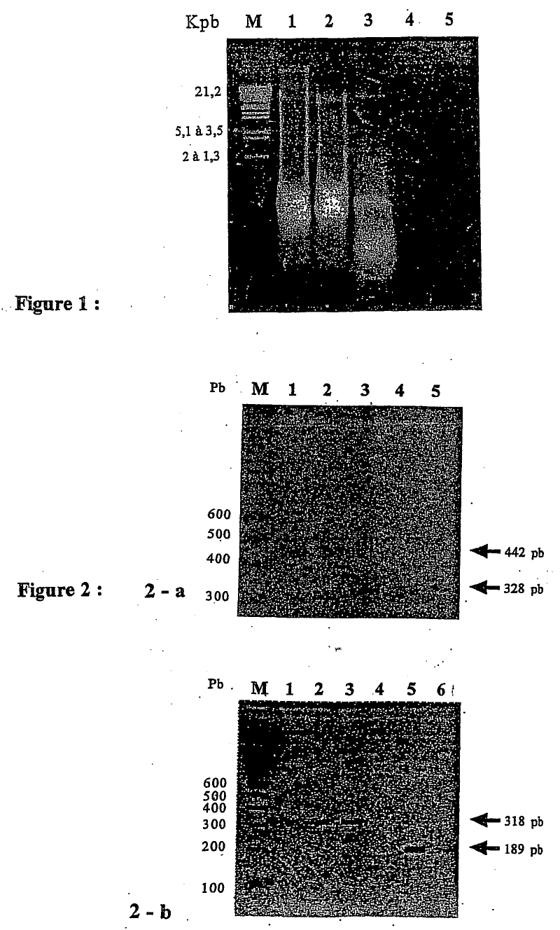

The migration of the DNAs originating from the various samples gives streaks characteristic of a degraded DNA, which is, however, perfectly amplifiable (FIG. 1).

2) Extraction of DNA by the Extraction Kit Method

This method is carried out in the conditions described by the supplier Qiagen.

### EXAMPLE 2

### Amplification of DNA

The PCR is carried out with the pair of primers SEQ ID N^{o}2 and SEQ ID N^{o}8 as defined above. The amplifications are realized in a total volume of 50 μl containing:

200 μg/ml of BSA (bovine serum albumin),

250 mM of dNTP (deoxynucleotide triphosphate),

300 ng of each primer,

1.5 mM of magnesium chloride (MgCl₂),

PCR 10× (100 mM Tris-HCl pH 8.3; 500 mM KCl) buffer,

1 unit of Taq Polymerase,

qsf 50 μl of sterile distilled water,

1 μl of DNA extract.

The reaction mixture is carried out in a sterile room under a horizontal-flux hood, in order to avoid contaminations as far as possible. The PCRs are carried out on an Ependorff PCR apparatus. Each PCR is divided up as follows:

1 initial cycle at a temperature of 94° C. for 2 minutes then,

40 cycles at a temperature of 94° C. for 1 minute, at a temperature of 55° C. to 63° C. for 1 minute, at a temperature of 72° C. for 2 minutes; in the last cycle a terminal elongation is carried out at a temperature of 72° C. for 7 minutes.

The amplification products are analysed by 2% agarose gel electrophoresis at a constant voltage of 100 V for 30 min, and using ethidium bromide for the visualization of the amplifications obtained.

The PCR reaction using the pair of primers SEQ ID N^{o}2 and SEQ ID N^{o}8 was carried out on a series of DNA extracts from fish. A single amplification product is observed, containing at least one DNA fragment SEQ ID N^{o}58 of a length of 442 base pairs (see wells 1 and 2 of FIG. 2-a), for the DNAs of gadiformes, in particular chosen from the group constituted by the gadidae, the merluccidae, the macrouridae and/or the moridae.

The PCR reaction using the pair of primers SEQ ID N^{o}5 and SEQ ID N^{o}8 was carried out on a series of DNA extracts from fish. A single amplification product is observed, containing at least one DNA fragment SEQ ID N^{o}59 of a length of 328 base pairs (see wells 3 and 4 of FIG. 2-a), for the DNAs of gadiformes, in particular chosen from the group constituted by the gadidae, the merluccidae, the macrouridae and/or the moridae (FIG. 2-a).

The PCR reaction using the pair of primers SEQ ID N^{o}20 and SEQ ID N^{o}26 was carried out on a series of DNA extracts from fish. A single amplification product is observed, containing at least one DNA fragment SEQ ID N^{o}62 of a length of 318 base pairs (see wells 2 and 3 of FIG. 2-b), for the DNAs of gadiformes, in particular chosen from the group constituted by the gadidae, the merluccidae, the macrouridae and/or the moridae.

The PCR reaction using the pair of primers SEQ ID N^{o}23 and SEQ ID N^{o}26 was carried out on a series of DNA extracts from fish. A single amplification product is observed, containing at least one DNA fragment SEQ ID N^{o}63 of a length of 189 base pairs (see wells 5 and 6 of FIG. 2-b), for the DNAs of gadiformes, in particular chosen from the group constituted by the gadidae, the merluccidae, the macrouridae and/or the moridae (FIG. 2-b).

The whole of the PCR product (amplification product) can be purified directly using the “QIAquick PCR Purification Kit” kit from Qiagen according to the stated conditions. The whole of the PCR product is passed over a column of silica gel. The nucleic acids are retained on the column whereas the other residues of the PCR are eluted by microcentrifugation. The impurities are eliminated and the DNA is eluted with Tris buffer or water. The thus-purified DNA is visualized and quantified on 2% agarose gel.

### EXAMPLE 3

### Characterization of the DNA Fragments by Amplification Followed by a Direct Sequencing (“Global Strategy”)

The automatic sequencing is carried out on the purified PCR products.

The primers used are those which served to amplify the DNA fragment or fragments that it is sought to characterize. The “Perkin-Elmer” kit is used for the PCR reaction, which is carried out over 25 cycles in the conditions stated by the supplier. Each amplification product obtained at the end of the PCR reaction is precipitated with ethanol and deposited on a polyacrylamide gel. The sequences obtained are then compared with those of the banks or with the sequences already obtained (FIG. 3).

### EXAMPLE 4

### Characterization of the DNA Fragments by Direct Amplification (“Specific Strategy”)

The PCR is carried out with the pairs of primers (SEQ ID N^{o}29 and SEQ ID N^{o}32) and (SEQ ID N^{o}41 and SEQ ID N^{o}44).

The amplifications are carried out in a total volume of 50 μl containing:

200 μg/ml of BSA,

250 mM of dNTP,

300 ng of each primer,

1.5 mM of MgCl₂,

PCR 10× (100 mM Tris-HCl pH 8.3; 500 mM KCl) buffer,

1 unit of Taq Polymerase,

qsf 50 μl of sterile distilled water,

1 μl of DNA extract

The reaction mixture is carried out in a sterile room under a horizontal-flux hood, in order to avoid contaminations as far as possible. The PCRs are carried out on an Ependorff PCR apparatus.

Each PCR is divided up as follows:

1 initial cycle at a temperature of 94° C. for 2 minutes then,

40 cycles at a temperature of 94° C. for 1 minute, at a temperature of approximately 60° C. to approximately 65° C. for 1 minute, at a temperature of 72° C. for 2 minutes; in the last cycle a terminal elongation is carried out at a temperature of 72° C. for 7 minutes.

The amplification product is analysed by 2% agarose gel electrophoresis at a constant voltage of 100 V for 30 min, and using ethidium bromide for the visualization of the amplification obtained (FIG. 4).

The use of the primers SEQ ID N^{o}29 and SEQ ID N^{o}32 allows an amplification product to be obtained containing a DNA fragment SEQ ID N^{o}64 which migrates forming a band of 168 base pairs, specific to Gadus morhua (FIG. 4-a).

The use of the primers SEQ ID N^{o}41 and SEQ ID N^{o}44 allows an amplification product to be obtained containing a DNA fragment SEQ ID N^{o}66 which migrates forming a band of 198 base pairs, specific to Theragra chalcogramma (FIG. 4-b).

### EXAMPLE 5

### Characterization of a Food Preparation Containing a Mixture of at Least Two Different Species of Gadiformes

According to an advantageous embodiment, the present invention allows the detection and identification of a mixture containing at least two different species of gadiformes. It is in fact common to prepare food preparations containing several species of fish (cooked dishes, soup, pates, terrine etc.). Thus, it may happen in particular that food preparations are constituted by a mixture of species containing a species of fish that is of less economic value compared with another species which should have been the only one found in the preparation (example: brandade of cod prepared with a little common or Atlantic cod (Gadus morhua), and a lot of Pacific cod (Gadus macrocephalus) which is less exploited and less expensive).

1) Global Strategy

When the sample of organic material to be analysed comprises a mixture of different species of fish, the amplification product obtained at the end of the PCR reaction contains several copies of different DNA fragments or sequences. However, when a sequencing of such an amplification product is carried out, a sequence is obtained which contains numerous indeterminations: it is therefore impossible to determine what species of fish are present in the sample of organic material to be analysed. According to an advantageous embodiment of the invention, a cloning of the amplification product obtained is then carried out: it is then possible to proceed with the sequencing of the different amplified DNA fragments thus separated from one another using the cloning.

The extraction and the amplification of the DNA contained in a sample of organic material containing a mixture of different species of fish is carried out in the way described above (see examples 1 and 2 above). At the end of the PCR reaction, a single amplification product is observed (containing at least one DNA fragment represented by SEQ ID N^{o}58 of a length of 442 base pairs), obtained using the primer pair (SEQ ID N^{o}2, SEQ ID N^{o}8) allowing amplification of all the species of gadiformes.

Said amplification product is therefore able to contain different DNA fragments characteristic of different species of gadiformes. In the present example, the amplification product (containing at least one DNA fragment represented by SEQ ID N^{o}58) contains different DNA fragments which it is sought to separate in order to be able to identify them. According to an advantageous embodiment of the invention, the use of a cloning method allows the separation of all the different DNA fragments contained in the amplification product, and thus the identification of each of these fragments (FIG. 7).

After purification of the amplification product (containing at least one DNA fragment represented by SEQ ID N^{o}58), this latter is cloned using the “TOPO TA cloning kit” system from Invitrogen according to the stated conditions, in order to isolate and obtain numerous identical copies of all the different DNA fragments contained in the amplification product. For this purpose, the amplification product is inserted by ligation in the “pCR®2.1—TOPO® 3.9 kb” plasmid marketed by Invitrogen, after cutting of said plasmid using a restriction enzyme: EcoR I (FIG. 7-1). Each DNA fragment contained in the amplification product will be inserted in a “pCR®2.1—TOPO®” plasmid (FIG. 7-2). A plasmid therefore corresponds to each DNA fragment. All the DNA fragments contained in the amplification product SEQ ID N^{o}58 are thus separated during this stage.

After ligation of each of the DNA fragments in a plasmid, each plasmid is introduced into a bacterium, for example Escherichia coli (E. coli) (FIG. 7-4), by a method called “transformation” which involves an osmotic shock and a temperature shock or electric shock. The E. coli bacteria thus obtained are cultured on gelose in order to be multiplied, in the presence of an antibiotic (for example ampicillin). The presence of the antibiotic allows the E. coli bacteria that have incorporated a plasmid to be selected, as the plasmids naturally possess a gene resistant to an antibiotic. Thus, the E. coli bacteria that have not incorporated a plasmid will not be able to develop.

A visual system is used to select the E. coli bacteria that have incorporated a plasmid in which a DNA fragment is ligated. In fact, two reagents (IPTG and X-Gal) were introduced into the gelose-containing medium which, on contact with the β-galactosidase enzyme, produce a blue colouring. The colonies of blue bacteria obtained are those which synthesize β-galactosidase, and which contain a plasmid in which a DNA fragment is not ligated. The colonies of white bacteria obtained are those which do not synthesize β-galactosidase, and which contain a plasmid in which a DNA fragment is ligated. This is linked with the fact that the DNA fragment is inserted in the plasmid by means of a gene coding for the β-galactosidase enzyme whose synthesis it blocks.

Each white bacterial colony obtained contains a single amplified DNA fragment or sequence, said DNA fragment or sequence corresponding to one and the same species of gadiformes. The plasmid DNAs containing the DNA fragments must then be recovered, i.e. separated from the bacterial DNAs in order to be sequenced. According to an advantageous embodiment of the method of the invention, some ten or so plasmid DNAs (symbolized respectively by the letters A, B, C, D, E, F, G, H, I and J on FIG. 7-8) are recovered, originating from 10 independent white bacterial colonies selected at random (represented by the letters A to J on FIG. 7-7). A bacterial culture is prepared from each white colony selected. The bacterial DNA is eliminated using the “QIAprep Spin Miniprep Kit” system from Qiagen according to the stated conditions. This purification aims essentially to eliminate the traces of the remaining bacterial DNA. It is carried out on resin which fixes the small DNA molecules (such as those of plasmid DNA), and not the bacterial DNA which is much longer. The plasmid DNA is fixed on the resin then eluted.

The 10 plasmid DNAs (symbolized by the letters A to J on FIG. 7-8) thus recovered are then sequenced with the primers supplied in the Invitrogen kit. The 10 sequences obtained are analysed so as to identify whether the profiles are different or not. The results obtained (FIG. 7-9) indicate that the sample of organic material comprises a mixture of two species, in this case Gadus morhua (fragments A, B, D, E, F, G, I and J) which is a fish belonging to the family of the gadidae, and Merluccius hubbsi (fragments C and H) which is a fish belonging to the family of the merluccidae.

Thus, if the DNA extracted from the sample of organic material to be analysed contains only a single species of gadiformes, a single sequence profile will be obtained for the 10 sampled and sequenced clones. The comparison of the obtained sequence with the reference sequences will allow identification of the species present in the sample of organic material.

If, on the other hand, the DNA extracted from the sample of organic material to be analysed is representative of several species of gadiformes, different profiles will be obtained corresponding respectively to the number of species present in the sample. The comparison of the different obtained sequences with the reference standards will allow identification respectively of the different species present in the sample of organic material.

It is important to note that the number of white bacterial colonies sampled can be increased if it is suspected that a large number of species are present in the sample of organic material to be analysed.

a) Detection and identification of a mixture of gadiformes: the Alaskan pollock (Theragra chalcogramma) belonging to the family of the gadidae, and the Argentine hake (Merluccius hubbsi) belonging to the family of the merluccidae.

The extraction of the DNA from a fish-based sample is carried out in the way described in Example 1 above.

The PCR is carried out with the primers SEQ ID N^{o}5 and SEQ ID N^{o}8 as defined above. A single amplification product is observed, containing at least one DNA fragment represented by SEQ ID N^{o}59 of a length of 328 base pairs, characteristic of DNA of gadiformes, in particular chosen from the group constituted by the gadidae, the merluccidae, the macrouridae and/or the moridae.

Said amplification product is purified, cloned then sequenced in the way described above. Two sequence profiles are observed on the 10 clones analysed. The interpretation of these two profiles shows that they correspond respectively to one species of gadidae: the Alaskan pollock (Theragra chalcogramma), and one species of merluccidae: the Argentine hake (Merluccius hubbsi).

b) Detection and identification of a mixture of gadidae: the Atlantic cod (Gadus morhua) and the ling (Molva molva).

The extraction of the DNA from a fish-based sample is carried out in the way described in Example 1 above.

The PCR is carried out with the primers SEQ ID N^{o}14 and SEQ ID N^{o}17 as defined above. A single amplification product is observed, containing at least one DNA fragment represented by SEQ ID N^{o}61 of a length of 237 base pairs, characteristic of DNA of gadidae.

Said amplification product is purified, cloned then sequenced in the way described above. Two sequence profiles are observed on the 10 clones analysed. The interpretation of these two profiles shows that they correspond respectively to two different species of gadidae: the Atlantic cod (Gadus morhua) and the ling (Molva molva).

c) Detection and identification of a mixture of merluccidae: the Cape hake (Merluccius capensis) and the common hake (Merluccius merluccius).

The extraction of the DNA from a fish-based sample is carried out in the way described in Example 1 above.

The PCR is carried out with the primers SEQ ID N^{o}23 and SEQ ID N^{o}26 as defined above. A single amplification product is observed, containing at least one DNA fragment represented by SEQ ID N^{o}63 of a length of 189 base pairs, characteristic of DNA of merluccidae.

Said amplification product is purified, cloned then sequenced in the way defined above. Two sequence profiles are observed on the 10 clones analysed. The interpretation of these two profiles shows that they correspond respectively to two different species of merluccidae: the Cape hake (Merluccius capensis) and the common hake (Merluccius merluccius).

2) Specific Strategy

In the case of the specific strategy, it is also possible to detect and identify a mixture of different species, without using cloning, since primers specific to 5 different species of gadidae are available. It is thus possible to detect and identify a mixture between precisely these 5 species of gadidae.

The example below illustrates the detection and the identification of a mixture of two species of gadidae: the Atlantic cod (Gadus morhua) and the Alaskan pollock (Theragra chalcogramma).

The extraction of the DNA from a fish-based sample is carried out in the way described in Example 1 above.

The PCR is carried out with successively the 5 pairs of primers as defined above for detection and direct identification, namely:

(SEQ ID N^{o}29 and SEQ ID N^{o}32),

(SEQ ID N^{o}35 and SEQ ID N^{o}38),

(SEQ ID N^{o}41 and SEQ ID N^{o}44),

(SEQ ID N^{o}47 and SEQ ID N^{o}50) and,

(SEQ ID N^{o}53 and SEQ ID N^{o}56).

Two amplification products are observed, containing respectively a DNA fragment represented by SEQ ID N^{o} 64 of a length of 168 base pairs, and a DNA fragment represented by SEQ ID N^{o} 66 of a length of 198 base pairs, characteristic of DNA of gadidae. On the other hand no amplification band is observed for the other wells (corresponding to the other primers).

Thus, it is deduced from this that the sample of organic material to be analysed contains Atlantic cod (Gadus morhua) and Alaskan pollock (Theragra chalcogramma).

1. Method for detecting the presence of biological materials originating from gadiformes chosen from the group constituted by the gadidae, the merluccidae, the macrouridae and/or the moridae, in a sample of organic material, characterized in that the presence of mitochondrial DNA originating from gadiformes chosen from the group constituted by the gadidae, the merluccidae, the macrouridae and/or the moridae in said organic material is determined by amplification of at least one sequence or fragment of mitochondrial DNA specific to the genome of the gadiformes chosen from the group constituted by the gadidae, the merluccidae, the macrouridae and/or the moridae, and contained in the mitochondrial DNA extracted from said sample, namely at least one sequence or fragment present in the genomes of the gadiformes chosen from the group constituted by the gadidae, the merluccidae, the macrouridae and/or the moridae, but absent from the genomes of the other animal genera, and in particular of the other animal species.

2. Method for detecting the presence of biological materials originating from gadiformes chosen from the group constituted by the gadidae, the merluccidae, the macrouridae and/or the moridae, in a sample of organic material, and for identifying the genus, in particular of at least one species of gadiformes chosen from the group constituted by the gadidae, the merluccidae, the macrouridae and/or the moridae, present in said sample, characterized in that the presence of mitochondrial DNA originating from gadiformes chosen from the group constituted by the gadidae, the merluccidae, the macrouridae and/or the moridae in said organic material is determined by amplification of at least one sequence or fragment of mitochondrial DNA specific to the genome of the gadiformes chosen from the group constituted by the gadidae, the merluccidae, the macrouridae and/or the moridae, and contained in the mitochondrial DNA extracted from said sample, namely at least one sequence or fragment present in the genomes of the gadiformes chosen from the group constituted by the gadidae, the merluccidae, the macrouridae and/or the moridae, but absent from the genomes of the other animal genera, in particular of the other animal species, and in that at least one sequence or fragment of mitochondrial DNA specific to the genome of the gadiformes chosen from the group constituted by the gadidae, the merluccidae, the macrouridae and/or the moridae, thus amplified, is compared with other mitochondrial DNA sequences of the genome of the gadiformes chosen from the group constituted by the gadidae, the merluccidae, the macrouridae and/or the moridae, said sequence of mitochondrial DNA or said fragment of mitochondrial DNA specific to the genome of the gadiformes chosen from the group constituted by the gadidae, the merluccidae, the macrouridae and/or the moridae, thus amplified, displaying at least approximately 50% identity, in particular approximately 60% identity with the other aforementioned sequences of mitochondrial DNA of the genome of the gadiformes chosen from the group constituted by the gadidae, the merluccidae, the macrouridae and/or the moridae.

3. Method according to claim 1, characterized in that it allows the detection and optionally the identification of the presence of gadidae, in particular chosen from the group constituted by Gadus morhua (common cod), Melanogrammus aeglefinus (haddock), Merlangius merlangus (whiting), Micromesistius poutassou (blue whiting), Pollachius virens (pollock), Pollachius pollachius (pollack), Trisopterus luscus (common pout), Trisopterus minutus capelanus (poor cod), Theragra chalcogramma (Alaskan pollock), Brome brome (tusk), Molva molva (ling) or Molva dypterygia dypterigia (blue ling).

4. Method according to claim 1, characterized in that it allows the detection and optionally the identification of the presence of merluccidae, in particular chosen from the group constituted by Merluccius albidus (offshore hake), Merluccius australis (Southern hake), Merluccius bilinearis (silver hake), Merluccius capensis (shallow-water Cape hake), Merluccius gayi (Chilean hake), Merluccius hubbsi (Argentine hake), Merluccius merluccius (common hake), Merluccius paradoxus (deep-water Cape hake), Merluccius productus (North Pacific hake), Merluccius senegalensis (Senegalese hake), Steindachneria argentea (silver hake).

5. Method according to claim 1, characterized in that the amplified sequence or fragment of the genome of the gadiformes chosen from the group constituted by the gadidae, the merluccidae, the macrouridae and/or the moridae, is situated in the central part of the gene coding for the cytochrome c oxidase of the mitochondrial DNA, delimited by the nucleotides situated in the vicinity of positions 6100 and 6601, and in particular in the vicinity of positions 6120 and 6590, and preferably in the vicinity of positions 6131 and 6580 of the gene coding for the cytochrome c oxidase of the mitochondrial DNA.

6. Oligonucleotides characterized in that that they are chosen from those:
(1)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}1 (positions 6131 to 6154 according to Johansen and Bakke, 1996, Molecular Marine Biology and Biotechnology, 5(3) 203-214):
TRT AYC ARC AYY TRT TYT GRT TCT K
in which R is A or G, Y is C or T, K is G or T,
on condition that the following sequences are excluded:
CGG GAT CCT GTT CTG ATT CTT GAT TTC C (SEQ ID NO: 69) and
CGA CGG GAT CCC AAC ACC TGT TTC GAT CAT CGC GGC AAC (SEQ ID NO: 70)
or comprising the following sequence SEQ ID N^{o}2 (positions 6134 to 6154 according to Johansen and Bakke, 1996, Molecular Marine Biology and Biotechnology, 5(3) 203-214):
AYC ARC AYY TRT TYT GRT TCT
in which Y is C or T, R is A or G,
or constituted by the following sequence SEQ ID N^{o}3 (positions 6139 to 6153 according to Johansen and Bakke, 1996, Molecular Marine Biology and Biotechnology, 5(3) 203-214):
AYY TRT TYT GRT TCT
in which Y is C or T, R is A or G,
or those
(2)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}4 (positions 6244 to 6269 according to Johansen and Bakke, 1996, Molecular Marine Biology and Biotechnology, 5(3) 203-214):
TTY GGN YAY ATR GGN ATR GTN TGA GC
in which Y is C or T, N is A, C, G or T, R is A or G,
or comprising the following sequence SEQ ID N^{o}5 (positions 6247 to 6269 according to Johansen and Bakke, 1996, Molecular Marine Biology and Biotechnology, 5(3) 203-214):
GGN YAY ATR GGN ATR GTN TGA GC
in which N is A, C, G or T, Y is C or T, R is A or G,
or constituted by the following sequence SEQ ID N^{o}6 (positions 6253 to 6269 according to Johansen and Bakke, 1996, Molecular Marine Biology and Biotechnology, 5(3) 203-214):
RGG NAT RGT NTG AGC
in which R is A or G, N is A, C, G or T,
or those
(3)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}7 (positions 6556 to 6580 according to Johansen and Bakke, 1996, Molecular Marine Biology and Biotechnology, 5(3) 203-214):
TAY GTW GTN GCN CAY TTY CAC TAC G
in which Y is C or T, W is A or T, N is A, C, G or T,
or comprising the following sequence SEQ ID N^{o}8 (positions 6556 to 6575 according to Johansen and Bakke, 1996, Molecular Marine Biology and Biotechnology, 5(3) 203-214):
TAY GTW GTN GCN CAY TTY CA
in which Y is C or T, W is A or T, N is A, C, G or T,
or constituted by the following sequence SEQ ID N^{o}9 (positions 6556 to 6570 according to Johansen and Bakke, 1996, Molecular Marine Biology and Biotechnology, 5(3) 203-214):
TAY GTW GTN GCN CAY
in which Y is C or T, W is A or T, N is A, C, G or T,
or those
(4)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}10 (positions 6131 to 6154 according to Johansen and Bakke, 1996, Molecular Marine Biology and Biotechnology, 5(3) 203-214):
TRT AYC ARC AYY TRT TYT GRT TCT K
in which R is A or G, Y is C or T, K is G or T,
or comprising the following sequence SEQ ID N^{o}11 (positions 6134 to 6154 according to Johansen and Bakke, 1996, Molecular Marine Biology and Biotechnology, 5(3) 203-214):
ACC AAC ACT TAT TCT GAT TCT
or constituted by the following sequence SEQ ID N^{o}12 (positions 6139 to 6154 according to Johansen and Bakke, 1996, Molecular Marine Biology and Biotechnology, 5(3) 203-214):
AYY AAC ACT TAT TCT
in which Y is C or T,
or those
(5)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}13 (positions 6277 to 6303 according to Johansen and Bakke, 1996, Molecular Marine Biology and Biotechnology, 5(3) 203-214):
CYA TYG GMC TYT YGG YTT TAT YGT V
in which Y is C or T, M is A or C, V is A, C or G,
or comprising the following sequence SEQ ID N^{o}14 (positions 6283 to 6303 according to Johansen and Bakke, 1996, Molecular Marine Biology and Biotechnology, 5(3) 203-214):
GGC CTC CTT GGC TTT ATT GTA
or constituted by the following sequence SEQ ID N^{o}15 (positions 6288 to 6303 according to Johansen and Bakke, 1996, Molecular Marine Biology and Biotechnology, 5(3) 203-214):
YTY GGY TTT ATT GTV
in which Y is C or T, V is A, C or G,
or those
(6)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}16 (positions 6496 to 6522 according to Johansen and Bakke, 1996, Molecular Marine Biology and Biotechnology, 5(3) 203-214):
GGM YTW ACA GGN ATY RTH YTR GCY AA
in which M is A or C, W is A or T, Y is C or T, N is A, C, G or T, R is A or G, H is A, C or T,
or comprising the following sequence SEQ ID N^{o}17 (positions 6496 to 6519 according to Johansen and Bakke, 1996, Molecular Marine Biology and Biotechnology, 5(3) 203-214):
GGC TTA ACA GGA ATT GTA CTA GCT
or constituted by the following sequence SEQ ID N^{o}18 (positions 6496 to 6510 according to Johansen and Bakke, 1996, Molecular Marine Biology and Biotechnology, 5(3) 203-214):
GGC TTA ACA GGA ATT
or those
(7)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}19 (positions 6195 to 6219 according to Johansen and Bakke, 1996, Molecular Marine Biology and Biotechnology, 5(3) 203-214):
CGG RAT AAT YTC YCA YAT YGT AGC C
in which R is A or G, Y is C or T,
or comprising the following sequence SEQ ID N^{o}20 (positions 6200 to 6219 according to Johansen and Bakke, 1996, Molecular Marine Biology and Biotechnology, 5(3) 203-214):
TAA TYT CYC AYA TYG TAG CC
in which Y is C or T,
or constituted by the following sequence SEQ ID N^{o}21 (positions 6205 to 6219 according to Johansen and Bakke, 1996, Molecular Marine Biology and Biotechnology, 5(3) 203-214):
TCY CAY ATY GTA GCC
in which Y is C or T,
or those
(8)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}22 (positions 6324 to 6348 according to Johansen and Bakke, 1996, Molecular Marine Biology and Biotechnology, 5(3) 203-214):
AGT BGG RAT RGA YGT DGA YAC MCG T
in which B is C, G or T, R is A or G, Y is C or T, D is A, G or T, M is A or C,
or comprising the following sequence SEQ ID N^{o}23 (positions 6329 to 6348 according to Johansen and Bakke, 1996, Molecular Marine Biology and Biotechnology, 5(3) 203-214):
GRA TRG AYG TDG AYA CMC GT
in which R is A or G, Y is C or T, D is A, G or T, M is A or C,
or constituted by the following sequence SEQ ID N^{o}24 (positions 6334 to 6348 according to Johansen and Bakke, 1996, Molecular Marine Biology and Biotechnology, 5(3) 203-214):
GAY GTD GAY ACM CGT
in which Y is C or T, D is A, G or T, M is A or C,
or those
(9)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}25 (positions 6498 to 6523 according to Johansen and Bakke, 1996, Molecular Marine Biology and Biotechnology, 5(3) 203-214):
ACT TAC AGG NAT YRT HCT RGC YAA YT
in which N is A, C, G or T, Y is C or T, R is A or G, H is A, C or T,
or comprising the following sequence SEQ ID N^{o}26 (positions 6498 to 6517 according to Johansen and Bakke, 1996, Molecular Marine Biology and Biotechnology, 5(3) 203-214):
ACT TAC AGG NAT YRT HCT RG
in which N is A, C, G or T, Y is C or T, R is A or G, H is A, C or T,
or constituted by the following sequence SEQ ID N^{o}27 (positions 6498 to 6512 according to Johansen and Bakke, 1996, Molecular Marine Biology and Biotechnology, 5(3) 203-214):
ACT TAC AGG NAT YRT
in which N is A, C, G or T, Y is C or T, R is A or G,
or those
(10)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}28 (positions 6399 to 6423 according to Johansen and Bakke, 1996, Molecular Marine Biology and Biotechnology, 5(3) 203-214):
AGT YTT YAG YTG AYT AGC AAC YYT V
in which Y is C or T, V is A, C or G,
or comprising the following sequence SEQ ID N^{o}29 (positions 6404 to 6423 according to Johansen and Bakke, 1996, Molecular Marine Biology and Biotechnology, 5(3) 203-214):
TTA GCT GAT TAG CAA CTT TA
or constituted by the following sequence SEQ ID N^{o}30 (positions 6409 to 6423 according to Johansen and Bakke, 1996, Molecular Marine Biology and Biotechnology, 5(3) 203-214):
TGA YTA GCA ACY YTV
in which Y is C or T, V is A, C or G,
or those
(11)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}31 (positions 6552 to 6577 according to Johansen and Bakke, 1996, Molecular Marine Biology and Biotechnology, 5(3) 203-214):
RTA YTA YGT AGT MGC YCA YTT YCA CT
in which R is A or G, Y is C or T, M is A or C,
or comprising the following sequence SEQ ID N^{o}32 (positions 6552 to 6572 according to Johansen and Bakke, 1996, Molecular Marine Biology and Biotechnology, 5(3) 203-214):
GTA TTA CGT AGT AGC CCA TT
or constituted by the following sequence SEQ ID N^{o}33 (positions 6552 to 6566 according to Johansen and Bakke, 1996, Molecular Marine Biology and Biotechnology, 5(3) 203-214):
RTA YTA YGT AGT MGC
in which R is A or G, Y is C or T, M is A or C,
or those
(12)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}34 (positions 6237 to 6261 according to Johansen and Bakke, 1996, Molecular Marine Biology and Biotechnology, 5(3) 203-214):
AGA RCC NTT YGG RYA YAT RGG HAT R
in which R is A or G, N is A, C, G or T, Y is C or T, H is A, C or T,
or comprising the following sequence SEQ ID N^{o}35 (positions 6242 to 6261 according to Johansen and Bakke, 1996, Molecular Marine Biology and Biotechnology, 5(3) 203-214):
CCT TTG GAT ATA TAG GCA TG
or constituted by the following sequence SEQ ID N^{o}36 (positions 6248 to 6261 according to Johansen and Bakke, 1996, Molecular Marine Biology and Biotechnology, 5(3) 203-214):
GGR YAY ATR GGH ATR
in which R is A or G, Y is C or T, H is A, C or T,
or those
(13)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}37 (positions 6381 to 6406 according to Johansen and Bakke, 1996, Molecular Marine Biology and Biotechnology, 5(3) 203-214):
YAT YCC RAC AGG YGT WAA AGT YTT YA
in which Y is C or T, R is A or G, W is A or T,
or comprising the following sequence SEQ ID N^{o}38 (positions 6381 to 6400 according to Johansen and Bakke, 1996, Molecular Marine Biology and Biotechnology, 5(3) 203-214):
TAT CCC AAC AGG TGT AAA AG
or constituted by the following sequence SEQ ID N^{o}39 (positions 6381 to 6395 according to Johansen and Bakke, 1996, Molecular Marine Biology and Biotechnology, 5(3) 203-214):
TAT YCC RAC AGG YGT
in which Y is C or T, R is A or G,
or those
(14)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}40 (positions 6267 to 6291 according to Johansen and Bakke, 1996, Molecular Marine Biology and Biotechnology, 5(3) 203-214):
AGC YAT RAT RGC YAT YGG MCT YCT Y
in which Y is C or T, R is A or G, M is A or C,
or comprising the following sequence SEQ ID N^{o}41 (positions 6272 to 6291 according to Johansen and Bakke, 1996, Molecular Marine Biology and Biotechnology, 5(3) 203-214):
TGA TGG CTA TTG GCC TCC TC
or constituted by the following sequence SEQ ID N^{o}42 (positions 6277 to 6291 according to Johansen and Bakke, 1996, Molecular Marine Biology and Biotechnology, 5(3) 203-214):
GCY ATY GGM CTY CTY
in which Y is C or T, M is A or C,
or those
(15)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}43 (positions 6451 to 6475 according to Johansen and Bakke, 1996, Molecular Marine Biology and Biotechnology, 5(3) 203-214):
HCC BMT MCT BTG RGC CCT V GG YTT YA
in which H is A, C or T, B is C, G or T, M is A or C, R is A or G, V is A, C or G, Y is C or T,
or comprising the following sequence SEQ ID N^{o}44 (positions 6451 to 6469 according to Johansen and Bakke, 1996, Molecular Marine Biology and Biotechnology, 5(3) 203-214):
CCC TCT ACT CTG AGC CCT AG
or constituted by the following sequence SEQ ID N^{o}45 (positions 6451 to 6464 according to Johansen and Bakke, 1996, Molecular Marine Biology and Biotechnology, 5(3) 203-214):
HCC BMT MCT BTG RGC
in which H is A, C or T, B is C, G or T, M is A or C, R is A or G,
or those
(16)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}46 (positions 6194 to 6219 according to Johansen and Bakke, 1996, Molecular Marine Biology and Biotechnology, 5(3) 203-214):
TSG RAT AAT YTC YCA YAT YGT AGC V
in which S is C or G, R is A or G, Y is C or T, V is A, C or G,
or comprising the following sequence SEQ ID N^{o}47 (positions 6200 to 6219 according to Johansen and Bakke, 1996, Molecular Marine Biology and Biotechnology, 5(3) 203-214):
TAA TTT CTC ACA TCG TAG CG
or constituted by the following sequence SEQ ID N^{o}48 (positions 6205 to 6219 according to Johansen and Bakke, 1996, Molecular Marine Biology and Biotechnology, 5(3) 203-214):
TCY CAY ATY GTA GCV
in which Y is C or T, V is A, C or G,
or those
(17)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}49 (positions 6342 to 6366 according to Johansen and Bakke, 1996, Molecular Marine Biology and Biotechnology, 5(3) 203-214):
YAC MCG WGC HTA CTT YAC ATC YGC A
in which Y is C or T, M is A or C, W is A or T, H is A, C or T
or comprising the following sequence SEQ ID N^{o}50 (positions 6342 to 6361 according to Johansen and Bakke, 1996, Molecular Marine Biology and Biotechnology, 5(3) 203-214):
TAC ACG TGC CTA CTT TAC AT
or constituted by the following sequence SEQ ID N^{o}51 (positions 6342 to 6356 according to Johansen and Bakke, 1996, Molecular Marine Biology and Biotechnology, 5(3) 203-214):
YAC MCG WGC HTA CTT
in which Y is C or T, M is A or C, W is A or T, H is A, C or T,
or those
(18)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}52 (positions 6152 to 6177 according to Johansen and Bakke, 1996, Molecular Marine Biology and Biotechnology, 5(3) 203-214):
TCT KCG GNC AYC CYG AAG THT AYA TH
in which K is G or T, N is A, C, G or T, Y is C or T, H is A, C or T,
or comprising the following sequence SEQ ID N^{o}53 (position 6158 to 6177 according to Johansen and Bakke, 1996, Molecular Marine Biology and Biotechnology, 5(3) 203-214):
GAC ACC CCG AAG TAT ACA TA
or constituted by the following sequence SEQ ID N^{o}54 (positions 6163 to 6177 according to Johansen and Bakke, 1996, Molecular Marine Biology and Biotechnology, 5(3) 203-214):
CCY GAA GTH TAY ATH
in which Y is C or T, H is A, C or T,
or those
(19)—displaying a sequence identity of at least 80%, preferably 90% and advantageously 95% with an oligonucleotide constituted by a sequence of approximately 15 to 25 nucleotides, in particular 20 to 25 nucleotides, comprised in the following sequence SEQ ID N^{o}55 (positions 6303 to 6328 according to Johansen and Bakke, 1996, Molecular Marine Biology and Biotechnology, 5(3) 203-214):
VTG RGC YCA YCA CAT RTT YAC AGT BG
in which V is A, C or G, R is A or G, Y is C or T, B is C, G or T,
or comprising the following sequence SEQ ID N^{o}56 (positions 6303 to 6322 according to Johansen and Bakke, 1996, Molecular Marine Biology and Biotechnology, 5(3) 203-214):
GTG AGC CCA TCA CAT GTT TA
or constituted by the following sequence SEQ ID N^{o}57 (positions 6303 to 6317 according to Johansen and Bakke, 1996, Molecular Marine Biology and Biotechnology, 5(3) 203-214):
VTG RGC YCA YCA CAT,
in which V is A, C or G, R is A or G, Y is C or T.

7. Pairs of primers characterized in that that they are constituted:
by any one of the oligonucleotides SEQ ID N^{o}1, SEQ ID N^{o}2, SEQ ID N^{o}3, SEQ ID N^{o}4, SEQ ID N^{o}5, SEQ ID N^{o}6, and any one of the oligonucleotides SEQ ID N^{o}7, SEQ ID N^{o}8, SEQ ID N^{o}9 according to claim 6 or,
by any one of the oligonucleotides SEQ ID N^{o}10, SEQ ID N^{o}11, SEQ ID N^{o}12, SEQ ID N^{o}13, SEQ ID N^{o}14, SEQ ID N^{o}15, and any one of the oligonucleotides SEQ ID N^{o}16, SEQ ID N^{o}17, SEQ ID N^{o}18 according to claim 6 or,
by any one of the oligonucleotides SEQ ID N^{o}19, SEQ ID N^{o}20, SEQ ID N^{o}21, SEQ ID N^{o}22, SEQ ID N^{o}23, SEQ ID N^{o}24, and any one of the oligonucleotides SEQ ID N^{o}25, SEQ ID N^{o}26, SEQ ID N^{o}27 according to claim 6 or,
by any one of the oligonucleotides SEQ ID N^{o}28, SEQ ID N^{o}29, SEQ ID N^{o}30, and any one of the oligonucleotides SEQ ID N^{o}31, SEQ ID N^{o}32, SEQ ID N^{o}33 according to claim 6 or,
by any one of the oligonucleotides SEQ ID N^{o}34, SEQ ID N^{o}35, SEQ ID N^{o}36, and any one of the oligonucleotides SEQ ID N^{o}37, SEQ ID N^{o}38, SEQ ID N^{o}39 according to claim 6 or,
by any one of the oligonucleotides SEQ ID N^{o}40, SEQ ID N^{o}41, SEQ ID N^{o}42, and any one of the oligonucleotides SEQ ID N^{o}43, SEQ ID N^{o}44, SEQ ID N^{o}45 according to claim 6 or,
by any one of the oligonucleotides SEQ ID N^{o}46, SEQ ID N^{o}47, SEQ ID N^{o}48, and any one of the oligonucleotides SEQ ID N^{o}49, SEQ ID N^{o}50, SEQ ID N^{o}51 according to claim 6 or,
by any one of the oligonucleotides SEQ ID N^{o}52, SEQ ID N^{o}53, SEQ ID N^{o}54, and any one of the oligonucleotides SEQ ID N^{o}55, SEQ ID N^{o}56, SEQ ID N^{o}57 according to claim 6,
and advantageously constituted by the pair of oligonucleotides chosen from the following pairs:
(SEQ ID N^{o}2 and SEQ ID N^{o}8),
(SEQ ID N^{o}5 and SEQ ID N^{o}8),
(SEQ ID N^{o}11 and SEQ ID N^{o}17),
(SEQ ID N^{o}14 and SEQ ID N^{o}17),
(SEQ ID N^{o}20 and SEQ ID N^{o}26),
(SEQ ID N^{o}23 and SEQ ID N^{o}26),
(SEQ ID N^{o}29 and SEQ ID N^{o}32),
(SEQ ID N^{o}35 and SEQ ID N^{o}38),
(SEQ ID N^{o}41 and SEQ ID N^{o}44),
(SEQ ID N^{o}47 and SEQ ID N^{o}50),
(SEQ ID N^{o}53 and SEQ ID N^{o}56).

8. Method according to claim 1, characterized in that the amplification of at least one sequence or fragment of mitochondrial DNA specific to the genome of the gadiformes chosen from the group constituted by the gadidae, the merluccidae, the macrouridae and/or the moridae, is carried out by the polymerase chain amplification method (PCR), comprising a repetition of the cycle of the following stages:
heating of the DNA extracted from the sample of organic material, so as to separate the DNA into two single-chain strands,
hybridization of oligonucleotide primers according to claims 6 and 7 or sequences CGG GAT CCT GTT CTG ATT CTT GAT TTC C (SEQ ID NO: 69) and CGA CGG GAT CCC AAC ACC TGT TTC GAT CAT CGC GGC AAC (SEQ ID NO: 70), to the monocatenary DNA strands at an adequate temperature, and
elongation of the oligonucleotide primers by a polymerase at an adequate temperature, in order to obtain at least one amplified DNA sequence or fragment specific to the genome of the gadiformes chosen from the group constituted by the gadidae, the merluccidae, the macrouridae and/or the moridae.

9. Method according to claim 1, characterized in that the obtained amplified mitochondrial DNA fragment(s) contained in the amplification product is (are) identified:
by sequencing of at least one amplified DNA fragment, and in particular of one amplified DNA fragment or,
directly, by visualization of the presence of the amplification product by gel electrophoresis.

10. Method according to claim 9, characterized in that the sequencing of each of the amplified DNA fragments is preceded by a cloning method when the sample of organic material comprises a mixture of different DNA fragments originating from different species of gadiformes chosen from the group constituted by the gadidae, the merluccidae, the macrouridae and/or the moridae, said cloning method permitting the separation from said mixture of the different DNA fragments originating from the different species of gadiformes.

11. Method according to claim 1, characterized in that the DNA extracted from the sample of organic material is:
non-degraded DNA originating in particular from a fresh sample or,
degraded DNA, originating in particular from a sample that has been transformed, in particular cooked, lyophilized, dried, pickled, appertized, pasteurized etc.

12. DNA fragment as amplified at the end of the method according to claim 1, characterized in that it comprises approximately 100 to approximately 500 base pairs.

13. DNA fragment according to claim 12, characterized in that it displays a sequence identity of at least 80%, preferably 90% and advantageously 95% with at least one of the sequences contained in:
the following SEQ ID N^{o}58:
AYCARCAYYT RTTCTGATTC TKCGGNCAYC CYGAAGTHTA
YATHCTNATY YTMCCHGGMT TCGGRATAAT YTCYCAYATY
GTAGCVTAYT AYTCAGGNAA RMAAGARCCN TTYGGRYAYA
TRGGHATRGT NTGAGCYATR ATRGCYATYG GMCTYCTYGG
YTTTATYGTV TGRGCYCAYC ACATRTTYAC AGTBGGRATR
GAYGTDGAYA CMCGWGCHTA CTTYACATCY GCAACBATAA
TYATYGCYAT YCCRACAGGY GTWAAAGTYT TYAGYTGAYT
AGCAACYYTV CAYGGRGGCT CARTTAARTG RGAVACHCCB
MTMCTBTGRG CCCTDGGYTT YATYTTYCTM TTYACMGTHG
GVGGMYTWAC AGGNATYRTH YTRGCYAAYT CYTCYCTAGA
YATYGTDCTY CAYGAYACRT AYTAMGTAGT MGCYCAYTTY
CA
in which Y is C or T, R is A or G, K is G or T, N is A, C, G or T, H is A, C or T, M is A or C, V is A, C or G, B is C, G or T, D is A, G or T, W is A or T,
said sequence SEQ ID N^{o} 58 comprising 442 base pairs,
or the following SEQ ID N^{o}59:
GGRYAYATRG GHATRGTNTG AGCYATRATR GCYATYGGMC
TYCTYGGYTT TATYGTVTGR GCYCAYCACA TRTTYACAGT
BGGRATRGAY GTDGAYACMC GWGCHTACTT YACATCYGCA
ACBATAATYA TYGCYATYCC RACAGGYGTW AAAGTYTTYA
GYTGAYTAGC ACYYTVCAYG GRGGCTCART TAARTGRGAV
ACHCCBMTMC TBTGRGCCCT DGGYTTYATY TTYCTMTTYA
CMGTHGGVGG MYTWACAGGN ATYRTHYTRG CYAAYTCYTC
YCTAGAYATY GTDCTYCAYG AYACRTAYTA MGTAGTMGCY
CAYTTYCA
in which R is A or G, Y is C or T, K is G or T, N is A, C, G or T, H is A, C or T, M is A or C, V is A, C or G, B is C, G or T, D is A, G or T, W is A or T,
said sequence SEQ ID N^{o} 59 comprising 328 base pairs,
or the following SEQ ID N^{o}60:

| | | | |
|---|---|---|---|
| AYCARCAYYT | RTTCTGATTC | TKCGGNCAYC | CYGAAGTHTA |
| YATHCTNATY | YTMCCHGGMT | TCGGRATAAT | YTCYCAYATY |
| GTAGCVTAYT | AYTCAGGNAA | RMAAGARCCN | TTYGGRYAYA |
| TRGGHATRGT | NTGAGCYATR | ATRGCYATYG | GMCTYCTYGG |
| YTTTATYGTV | TGRGCYCAYC | ACATRTTYAC | AGTBGGRATR |
| GAYGTDGAYA | CMCGWGCHTA | CTTYACATCY | GCAACBATAA |
| TYATYGCYAT | YCCRACAGGY | GTWAAAGTYT | TYAGYTGAYT |
| AGCAACYYTV | CAYGGRGGCT | CARTTAARTG | RGAVACHCCB |
| MTMCTBTGRG | CCCTDGGYTT | YATYTTYCTM | TTYACMGTHG |
| GVGGMYTWAC | AGGNATYRTH | YTRGCY | |

in which Y is C or T, R is A or G, K is G or T, N is A, C, G or T, H is A, C or T, M is A or C, V is A, C or G, B is C, G or T, D is A, G or T, W is A or T,
said sequence SEQ ID N^{o}60 comprising 386 base pairs,
or the following SEQ ID N^{o}61:
GGMCTYCTYG GYTTTATYGT VTGRGCYCAY CACATRTTYA
CAGTBGGRAT RGAYGTDGAY ACMCGWGCHT ACTTYACATC
YGCAACBATA ATYATYGCYA TYCCRACAGG YGTWAAAGTY
TTYAGYTGAY TAGCAACYYT VCAYGGRGGC TCARTTAART
GRGAVACHCC BMTMCTBTGR GCCCTDGGYT TYATYTTYCT
MTTYACMGTH GGVGGMYTWA CAGGNATYRT HYTRGCY
in which Y is C or T, R is A or G, K is G or T, N is A, C, G or T, H is A, C or T, M is A or C, V is A, C or G, B is C, G or T, D is A, G or T, W is A or T,
said sequence SEQ ID N^{o} 61 comprising 237 base pairs,
or the following SEQ ID N^{o}62:

| | | | |
|---|---|---|---|
| TAATYTCYCA | YATYGTAGCV | TAYTAYTCAG | GNAARMAAGA |
| RCCNTTYGGR | YAYATRGGHA | TRGTNTGAGC | YATRATRGCY |
| ATYGGMCTYC | TYGGYTTTAT | YGTVTGRGCY | CAYCACATRT |
| TYACAGTBGG | RATRGAYGTD | GAYACMCGWG | CHTACTTYAC |
| ATCYGCAACB | ATAATYATYG | CYATYCCRAC | AGGYGTWAAA |
| GTYTTYAGYT | GAYTAGCAAC | YYTVCAYGGR | GGCTCARTTA |
| ARTGRGAVAC | HCCBMTMCTB | TGRGCCCTDG | GYTTYATYTT |
| YCTMTTYACM | GTHGGVGGMY | TWACAGGNAT | YRTHYTRG |

in which Y is C or T, R is A or G, K is G or T, N is A, C, G or T, H is A, C or T, M is A or C, V is A, C or G, B is C, G or T, D is A, G or T, W is A or T,
said sequence SEQ ID N^{o} 62 comprising 318 base pairs,
or the following SEQ ID N^{o}63:

| | | | |
|---|---|---|---|
| GRATRGAYGT | DGAYACMCGW | GCHTACTTYA | CATCYGCAAC |
| BATAATYATY | GCYATYCCRA | CAGGYGTWAA | AGTYTTYAGY |
| TGAYTAGCAA | CYYTVCAYGG | RGGCTCARTT | AARTGRGAVA |
| CHCCBMTMCT | BTGRGCCCTD | GGYTTYATYT | TYCTMTTYAC |
| MGTHGGVGGM | YTWACAGGNA | TYRTHYTRG | |

in which R is A or G, Y is C or T, D is A, G or T, M is A or C, W is A or T, B is C, G or T, V is A, C or G, H is A, C or T, N is A, C, G or T,
said sequence SEQ ID N^{o}63 comprising 189 base pairs,
or the following SEQ ID N^{o}64:

| | | | |
|---|---|---|---|
| TYAGYTGAYT | AGCAACYYTV | CAYGGRGGCT | CARTTAARTG |
| RGAVACHCCB | MTMCTBTGRG | CCCTDGGYTT | YATYTTYCTM |
| TTYACMGTHG | GVGGMYTWAC | AGGNATYRTH | YTRGCYAAYT |
| CYTCYCTAGA | YATYGTDCTY | CAYGAYACRT | AYTAMGTAGT |
| MGCYCAYT | | | |

in which Y is C or T, V is A, C or G, R is A or G, B is C, G or T, H is A, C or T, M is A or C, D is A, G or T, N is A, C, G or T,
said sequence SEQ ID N^{o}64 comprising 168 base pairs,
or the following SEQ ID N^{o}65:

| | | | |
|---|---|---|---|
| CNTTYGGRYA | YATRGGHATR | GTNTGAGCYA | TRATRGCYAT |
| YGGMCTYCTY | GGYTTTATYG | TVTGRGCYCA | YCACATRTTY |
| ACAGTBGGRA | TRGAYGTDGA | YACMCGWGCH | TACTTYACAT |
| CYGCAACBAT | AATYATYGCY | ATYCCRACAG | GYGTWAAAG |

in which N is A, C, G or T, R is A or G, Y is C or T, H is A, C or T, M is A or C, V is A, C or G, B is C, G or T, D is A, G or T, W is A or T,
said sequence comprising 159 base pairs,
or the following sequence SEQ ID N^{o}66:

| | | | |
|---|---|---|---|
| TRATRGCYAT | YGGMCTYCTY | GGYTTTATYG | TVTGRGCYCA |
| YCACATRTTY | ACAGTBGGRA | TRGAYGTDGA | YACMCGWGCH |
| TACTTYACAT | CYGCAACBAT | AATYATYGCY | ATYCCRACAG |
| GYGTWAAAGT | YTTYAGYTGA | YTAGCAACYY | TVCAYGGRGG |
| CTCARTTAAR | TGRGAVACHC | CBMTMCTBTG | RGCCCTDG |

in which R is A or G, Y is C or T, M is A or C, V is A, C or G, B is C, G or T, D is A, G or T, W is A or T, H is A, C or T.
said SEQ ID N^{o}66 comprising 198 base pairs,
the following SEQ ID N^{o}67:

| | | | |
|---|---|---|---|
| TAATYTCYCA | YATYGTAGCV | TAYTAYTCAG | GNAARMAAGA |
| RCCNTTYGGR | YAYATRGGHA | TRGTNTGAGC | YATRATRGCY |
| ATYGGMCTYC | TYGGYTTTAT | YGTVTGRGCY | CAYCACATRT |
| TYACAGTBGG | RATRGAYGTD | GAYACMCGWG | CHTACTTYAC |
| AT | | | |

in which Y is C or T, V is A, C or G, N is A, C, G or T, R is A or G, M is A or C, H is A, C or T, B is C, G or T, D is A, G or T, W is A or T,
said sequence SEQ ID N^{o}67 comprising 162 base pairs,
or the following SEQ ID N^{o}68:

| | | | |
|---|---|---|---|
| GNCAYCCYGA | AGTHTAYATH | CTNATYYTMC | CHGGMTTCGG |
| RATAATYTCY | CAYATYGTAG | CVTAYTAYTC | AGGNAARMAA |
| GARCCNTTYG | GRYAYATRGG | HATRGTNTGA | GCYATRATRG |
| CYATYGGMCT | YCTYGGYTTT | ATYGTVTGRG | CYCAYCACAT |
| RTTYA | | | |

in which N is A, C, G or T, Y is C or T, H is A, C or T, M is A or C, R is A or G, V is A, C or G,
said sequence SEQ ID NO 68 comprising 165 base pairs.

14. Method according to claim 1, characterized in that the presence of mitochondrial DNA originating from gadiformes in a sample of organic material is detected by amplification of at least one sequence of mitochondrial DNA specific to the genome of the gadiformes using any one of the oligonucleotides SEQ ID N^{o}1, SEQ ID N^{o}2, SEQ ID N^{o}3, SEQ ID N^{o}4, SEQ ID N^{o}5, SEQ ID N^{o}6, and any one of the oligonucleotides SEQ ID N^{o}7, SEQ ID N^{o}8, SEQ ID N^{o}9 as defined in claim 6,
and advantageously using the pair of oligonucleotides (SEQ ID N^{o}2 and SEQ ID N^{o}8) or the pair of oligonucleotides (SEQ ID N^{o}5 and SEQ ID N^{o}8), in order to obtain respectively at least one of the DNA sequences contained in SEQ ID N^{o}58 or in SEQ ID N^{o}59 as defined in claim 13, said sequences being specific to the genome of the gadiformes,
and in that at least one species of gadiforme present in said sample of organic material is identified by sequencing of at least one of the DNA sequences contained in SEQ ID N^{o}58 or in SEQ ID N^{o}59.

15. Method according to any claim 1, characterized in that the presence of mitochondrial DNA originating from gadidae in a sample of organic material is detected by amplification of at least one sequence of mitochondrial DNA specific to the genome of the gadidae using any one of the oligonucleotides SEQ ID N^{o}10, SEQ ID N^{o}11, SEQ ID N^{o}12, SEQ ID N^{o}13, SEQ ID N^{o}14, SEQ ID N^{o}15, and any one of the oligonucleotides SEQ ID N^{o}16, SEQ ID N^{o}17, SEQ ID N^{o}18 as defined in claim 6,
and advantageously using the pair of oligonucleotides (SEQ ID N^{o}11 and SEQ ID N^{o}17) or the pair of oligonucleotides (SEQ ID N^{o}14 and SEQ ID N^{o}17), in order to obtain respectively at least one of the DNA sequences contained in SEQ ID N^{o}60 or in SEQ ID N^{o}61 as defined in claim 13, said sequences being specific to the genome of the gadidae,
and in that at least one species of gadidae present in said sample of organic material is identified by sequencing of at least one of the DNA sequences contained in SEQ ID N^{o}60 or in SEQ ID N^{o}61.

16. Method according to claim 1, characterized in that the presence of DNA originating from merluccidae in a sample of organic material is detected by amplification of at least one sequence of mitochondrial DNA specific to the genome of the merluccidae using any one of the oligonucleotides SEQ ID N^{o}19, SEQ ID N^{o}20, SEQ ID N^{o}21, SEQ ID N^{o}22, SEQ ID N^{o}23, SEQ ID N^{o}24, and any one of the oligonucleotides SEQ ID N^{o}25, SEQ ID N^{o}26, SEQ ID N^{o}27 as defined in claim 6,
and advantageously using the pair of oligonucleotides (SEQ ID N^{o}20 and SEQ ID N^{o}26) or the pair of oligonucleotides (SEQ ID N^{o}23 and SEQ ID N^{o}26), in order to obtain respectively at least one of the DNA sequences contained in SEQ ID N^{o}62 or in SEQ ID N^{o}63 as defined in claim 13, said sequences being specific to the genome of the merluccidae,
and in that at least one species of merluccidae present in said sample of organic material is identified by sequencing of at least one of the DNA sequences contained in SEQ ID N^{o}62 or in SEQ ID N^{o}63.

17. Method according to claim 1, characterized in that the presence of mitochondrial DNA originating from gadiformes is identified in a sample of organic material, and in particular of gadidae chosen from the group constituted by the species Gadus morhua (common cod), Pollachius virens (pollock), Theragra chalcogramma (Alaskan pollock), Melanogrammus aeglefinus (haddock) and Merlangius merlangus (whiting), and in that each of the aforementioned species is identified by amplification of at least one DNA sequence specific to the genome of each of the aforementioned species of gadidae, with the help respectively:
of any one of the oligonucleotides SEQ ID N^{o}28, SEQ ID N^{o}29, SEQ ID N^{o}30, and any one of the oligonucleotides SEQ ID N^{o}31, SEQ ID N^{o}32, SEQ ID N^{o}33 as defined in claim 6 or,
of any one of the oligonucleotides SEQ ID N^{o}34, SEQ ID N^{o}35, SEQ ID N^{o}36, and any one of the oligonucleotides SEQ ID N^{o}37, SEQ ID N^{o}38, SEQ ID N^{o}39 as defined in claim 6 or,
of any one of the oligonucleotides SEQ ID N^{o}40, SEQ ID N^{o}41, SEQ ID N^{o}42, and any one of the oligonucleotides SEQ ID N^{o}43, SEQ ID N^{o}44, SEQ ID N^{o}45 as defined in claim 6 or,
of any one of the oligonucleotides SEQ ID N^{o}46, SEQ ID N^{o}47, SEQ ID N^{o}48, and any one of the oligonucleotides SEQ ID N^{o}49, SEQ ID N^{o}50, SEQ ID N^{o}51 as defined in claim 6 or,
of any one of the oligonucleotides SEQ ID N^{o}52, SEQ ID N^{o}53, SEQ ID N^{o}54, and any one of the oligonucleotides SEQ ID N^{o}55, SEQ ID N^{o}56, SEQ ID N^{o}57 as defined in claim 6,
and advantageously using respectively:
the pair of oligonucleotides (SEQ ID N^{o}29 and SEQ ID N^{o}32) or,
the pair of oligonucleotides (SEQ ID N^{o}35 and SEQ ID N^{o}38) or,
the pair of oligonucleotides (SEQ ID N^{o}41 and SEQ ID N^{o}44) or,
the pair of oligonucleotides (SEQ ID N^{o}47 and SEQ ID N^{o}50) or,
the pair of oligonucleotides (SEQ ID N^{o}53 and SEQ ID N^{o}56),
in order to obtain respectively at least one of the DNA sequences contained in:
SEQ ID N^{o}64 specific to the genome of Gadus morhua (common cod),
SEQ ID N^{o}65 specific to the genome of Pollachius virens (pollock),
SEQ ID N^{o}66 specific to the genome of Theragra chalcogramma (Alaskan pollock),
SEQ ID N^{o}67 specific to the genome of Melanogrammus aeglefinus (haddock),
SEQ ID N^{o}68 specific to the genome of Merlangius merlangus (whiting),
said SEQ ID N^{o}64, SEQ ID N^{o}65, SEQ ID N^{o}66, SEQ ID N^{o}67 and SEQ ID N^{o}68.

18. A method for detecting the presence of biological materials originating from gadiformes, comprising screening for nucleotide sequences chosen from the oligonucleotide primers according to claim 6 or from sequences CGG GAT CCT GTT CTG ATT CTT GAT TTC C (SEQ ID NO: 69) and CGA CGG GAT CCC AAC ACC TGT TTC GAT CAT CGC GGC AAC (SEQ ID NO: 70).
